(19) United States
(12) Patent Application Publication
(10) Pub. No.: US 2022/0267367 A1
(43) Pub. Date: Aug. 25, 2022

(54) COMPOSITIONS FOR THE PREVENTION AND TREATMENT OF PARKINSON'S DISEASE

(71) Applicant: Metselex, Inc., Minneapolis, MN (US)

(72) Inventors: Walter Low, Shorewood, MN (US); Clifford Steer, Apple Valley, MN (US); Michael D. Finch, Apple Valley, MN (US); Cyrus B. Munshi, Blaine, MN (US)

(21) Appl. No.: 17/703,877

(22) Filed: Mar. 24, 2022

Related U.S. Application Data
(63) Continuation of application No. 17/041,720, filed on Sep. 25, 2020, now abandoned.
(60) Provisional application No. 62/649,892, filed on Mar. 29, 2018.

(51) Int. Cl. C07J 9/00; C07J 41/00; C07J 63/00; C07J 71/00

(52) U.S. Cl. CPC C07J 9/005; C07J 41/0061; C07J 63/008; C07J 71/0005

(57) ABSTRACT

Methods of preventing or retarding or reversing or abolishing the onset of Parkinson's and other neurodegenerative diseases are discussed.

This application is a continuation of U.S. application Ser. No. 17/041,720, filed Sep. 25, 2020, which is a U.S. national stage filing under 35 U.S.C. § 371 from International Application No. PCT/US2019/024922, filed on Mar. 29, 2019, and published as WO2019/191637 A1 on Oct. 3, 2019, which claims the benefit of priority to U.S. Provisional Patent Application No. 62/649,892, filed on Mar. 29, 2018, the contents of all of which are specifically incorporated by reference herein in their entirety.

### BACKGROUND OF THE INVENTION

### Parkinson's Disease:

Parkinson's disease (PD) is a neurodegenerative disorder characterized by the selective loss of dopaminergic neurons in the substantia nigra of brain. Although there are multiple pathogenic mechanisms in PD, the most common postulated pathogenic mechanism in PD is a vicious cycle of oxidative stress. Postmortem studies showed that oxidative damage and decrease in anti-oxidative glutathione in PD brain tissues, and multiple signs of apoptosis, such as mitochondrial dysfunction, chromatin condensation, and caspase activation in dying cells. For these reasons, much interest has focused on the antioxidant and anti-apoptotic defenses that may be promising therapeutics for PD. Unknown at this juncture are the underlying causes of PD, although it is believed to result from a combination of genetic predisposition and a possible external stimulus. The general symptoms of PD are triggered by a severe loss of dopamine production in the substantia nigra. Along these lines, it has been shown that in a small number of clinical patients with PD who are also recipients of transplanted human embryonic dopamine neurons, return to a normal life. A minimum of 80,000 dopamine-producing neurons is required for benefits from any clinical intervention, greatly accentuating the need for enhanced life of transplanted neurons.

### SUMMARY OF THE INVENTION

The present invention describes a method of preventing or delaying the onset of or abolishing Parkinson's and related diseases by preventing cell death of neurological tissue. The patient is a human patient, while the administering step involves administering, through various means, an amount of UDCA or TUDCA, in any formulation in any combination that is effective in providing the necessary pharmacological benefit.

One feature of the present invention involves the administering of an effective amount of phosphorylated dopaminergic prodrugs of bile acids or any of their analogs or formulations or any combination thereof. The mode of administering these prodrugs includes, but is not limited to, intravenously, parenterally, orally or intramuscularly or any combination of these methods thereof.

Another feature of the invention involves the administering of an effective amount of these prodrugs or any of their analogs or derivatives.

Herein, a “patient” includes a human or any mammal.

### BRIEF DESCRIPTION OF THE FIGURES

FIG. 1. TUDCA prevents Bax-induced alterations in mitochondrial membrane polarity.

FIG. 2. TUDCA prevents Bax-induced alterations in mitochondrial membrane protein order.

FIG. 3A-D. UDCA reduces apoptosis in dopaminergic SH-SYSY neuronal cell line.

FIG. 4A-B. UDCA significantly impacts the protein levels of Bax and Bcl-2 and cytochrome c in dopaminergic SH-SYSY neuronal cell line.

FIG. 5. Alkaline-phosphatase-activation of phosphorylated UDCA.

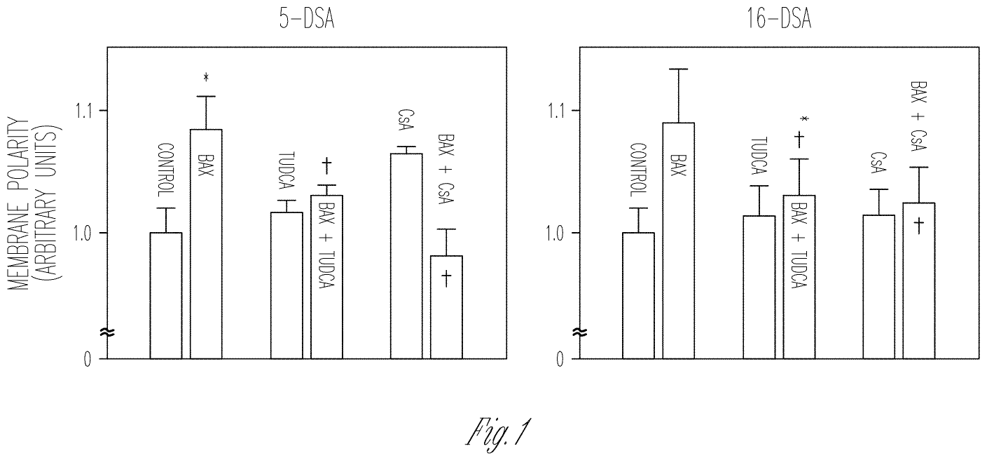

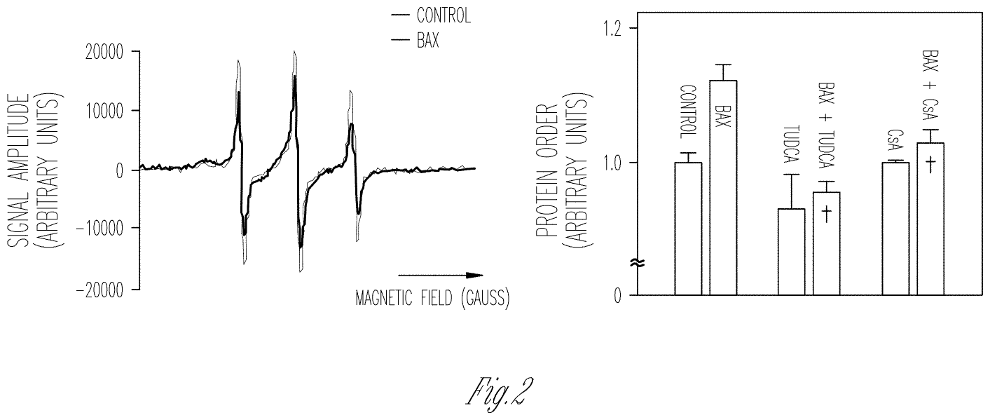

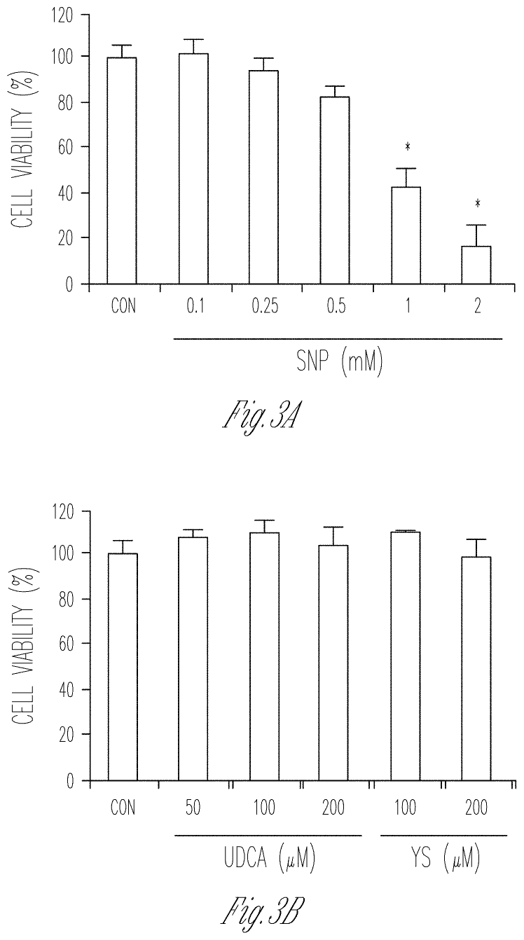

### DETAILED DESCRIPTION OF THE INVENTION

The current invention describes a method of treating a patient exhibiting symptoms of several neurodegenerative diseases including Parkinson's disease.

Described here in, inter ilia, are compositions and preparations of DOPA and L-DOPA analogs of phosphorylated bile acid prodrugs.

The invention provides in some embodiments a compound having the phosphorylated prodrug formula (I):

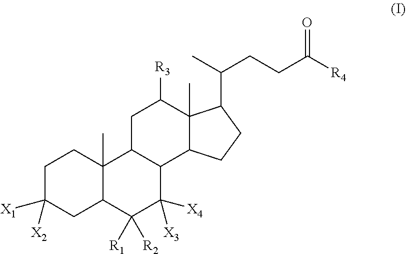

### Wherein

R1 is —OH, or (PO4), -L-Dopa, -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine.
R2 is —OH, or (PO4), -L-Dopa, or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R3 is —OH, —H or (PO4)
R4 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
X1 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
X2 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
X3 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
X4 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine.
   Or a compound having the formula (II)

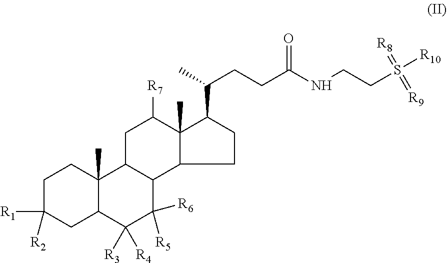

### Wherein

R1 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R2 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R3 is —OH, —H or —(PO4)
R4 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R5 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R6 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R7 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R8 is —H, —OH, —(PO4), ═O, -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R9 is —H, —OH, —(PO4), ═O, -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R10 is —H, —OH, —(PO4), —OH, -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
   Or a compound having the formula (III)

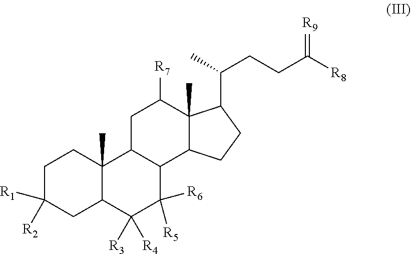

### Wherein

R1 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R2 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R3 is —OH, —H or (PO4)
R4 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R5 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R6 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R7 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R8 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R9 is —H, —OH, —(PO4), -L-Dopa or -Dopa -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
   Or a compound having the formula (IV)

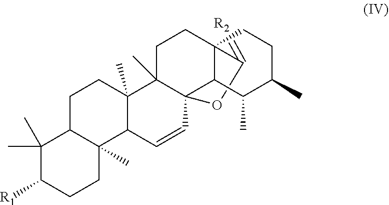

### Wherein

R1 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R2 is —OH, ═O, (PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine

Or a compound having the formula (V)

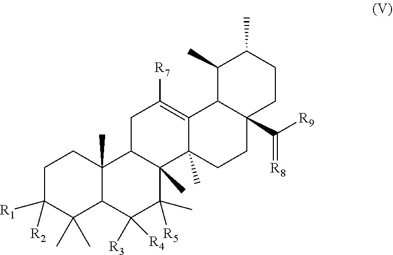

Wherein
R1 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R2 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R3 is —OH, —H or (PO4)
R4 is —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R5 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R6 is —H, —OH, —(PO4), -L-Dopa or Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R7 is —H, —OH, —(PO4), -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R8 is —H, —OH, —(PO4), ═O, —OH, -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine
R9 is —H, —OH, —(PO4), —OH, -L-Dopa or -Dopa, -alkyl-L-Dopa, or alkyl-Dopa, or monoamine oxidase inhibitor (MAO), or catechol-O-methyl transferase (COMT), or monoamine re-uptake inhibitors, or glutamate receptor antagonists, or lipoic acid, or acetyl-L-carnitine

### DETAILED DESCRIPTION OF THE INVENTION

Ursodeoxycholic acid has two alcohol moieties to which a phosphate can be directly attached, located at the 3- and 7-positions. We began our synthesis of both of these potential prodrugs by benzyl protecting the acid of UDCA, which proceeded in high yield using benzyl bromide as the alkylating agent (Scheme 1). Heating the resulting benzyl ester 1 with dibenzyl N,N-diethylphosphoramidite followed by oxidation with

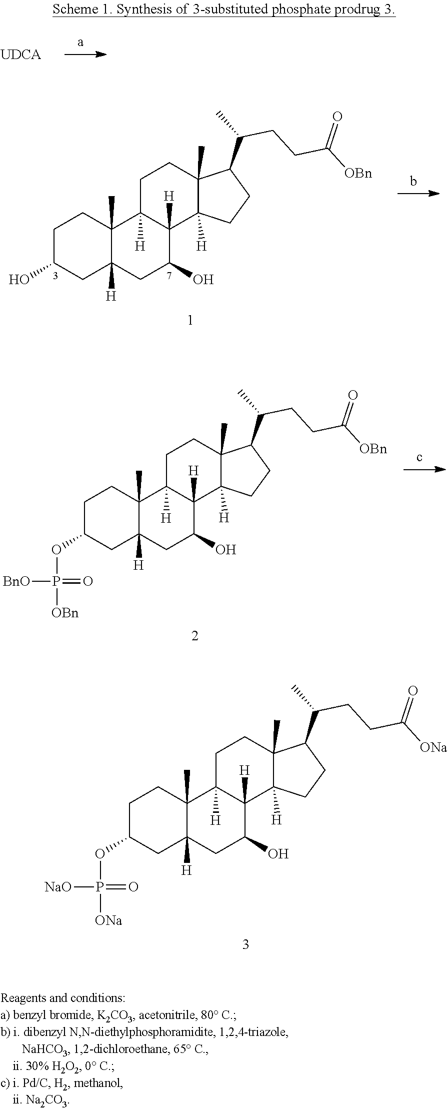

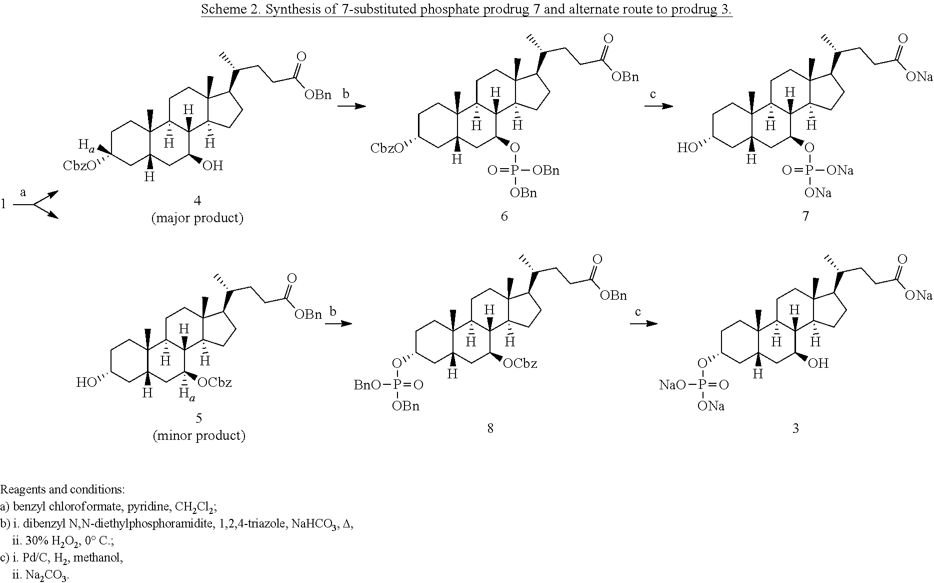

H₂O₂ furnished a phosphate ester which was tentatively assigned the structure 2 based on reports that similar steroidal structures react more readily at the 3-position than at the 7-position. This assignment was later confirmed by NMR spectroscopy (see below). Removal of the three benzyl groups of 2 using hydrogen and Pd/C followed by treatment with sodium carbonate yielded the desired 3-substituted phosphate ester prodrug of UDCA (3).

To obtain the 7-substituted phosphate ester prodrug of UDCA we treated benzyl ester 1 with benzyl chloroformate and pyridine in dichloromethane (Scheme 2). This led to a mixture of products, including 3-Cbz-protected alcohol 4 (41%), 7-Cbz-protected alcohol 5 (10%), recovered starting material (41%) and a small amount of 3,7-diCbz-protected material. These products could readily be separated by column chromatography and allowed us to unambiguously confirm the regiochemistry of our prodrugs by ¹H NMR analysis, as the signal of the proton next to the Cbz-protected alcohol (Hₐ in Scheme 2) was a dddd in the major mono-substituted product (consistent with structure 4) and a ddd in the minor mono-substituted product (consistent with structure 5). Compound 4 was then converted into 7-substituted phosphate ester prodrug 7 using dibenzyl N,N-diethylphosphoramidite followed by oxidation with H₂O₂ and then Pd/C catalyzed debenzylation. Similar standard conditions converted 5 into the same 3-substituted phosphate ester prodrug 3 that was obtained using Scheme 1. Both phosphate prodrugs were highly aqueously soluble, rapidly dissolving at all concentrations tested (up to 20 mg/mL), and were stable in solution for extended periods of time (>6 months) without any apparent decomposition.

In addition to prodrugs 3 and 7, where the phosphate is directly linked to one of the alcohols in UDCA, we also set out to synthesize 3- and 7-substituted oxymethylphosphate (OMP) UDCA prodrugs. While often more difficult to synthesize, OMP prodrugs (also referred to a phosphonooxymethyl or POM prodrugs) are typically bioactivated by alkaline phosphatase at a significantly faster rate than their directly linked phosphate ester analogs due to reduced steric hindrance, which would be preferred for rapid treatment of stroke or myocardial infarction. Upon bioactivation, OMP prodrugs release parent drug and formaldehyde in a two-step process.

The synthesis of the 3- and 7-substituted oxymethylphosphate (OMP) prodrugs of UDCA did indeed prove to be considerably more complicated than the synthesis of the directly linked phosphate prodrugs 3 and 7. Attempts to directly alkylate the UDCA scaffold at either the 3- or 7-positions with either dibenzyl chloromethyl phosphate or chloroiodomethane were unsuccessful. Instead, we turned to a synthetic scheme that had previously be used to synthesize OMP prodrugs, namely methylthiomethyl (MTM) ether formation followed by reaction with N-iodosuccinimide (NIS) and a phosphate. We successfully synthesized the desired MTM ether intermediate 9 via a Pummerer rearrangement by stirring 4 in DMSO, acetic anhydride, and acetic acid (Scheme 3). Unfortunately, treating 9 with NIS and either dibenzyl phosphate or H₃PO₄ did not lead to any isolable product. However, we were able to convert the MTM ether 9 into chloroalkyl ether 11 by heating it in CH₂Cl₂ and thionyl chloride. In an NMR experiment, reaction of chloroalkyl ether 11 with dibenzyl phosphate and K₂CO₃ in acetonitrile-d₃ led to an impure product (likely 10) which decomposed before it could be isolated. Similarly, reaction of 11 with either K₃PO₄ or Na₃PO₄ failed to lead to the desired OMP product.

We were finally able to successfully substitute 11 by following the example of Komatatsu and coworkers, who found that a tri(n-butyl)amine salt of phosphate could be successfully reacted with a chloroalkyl ether:

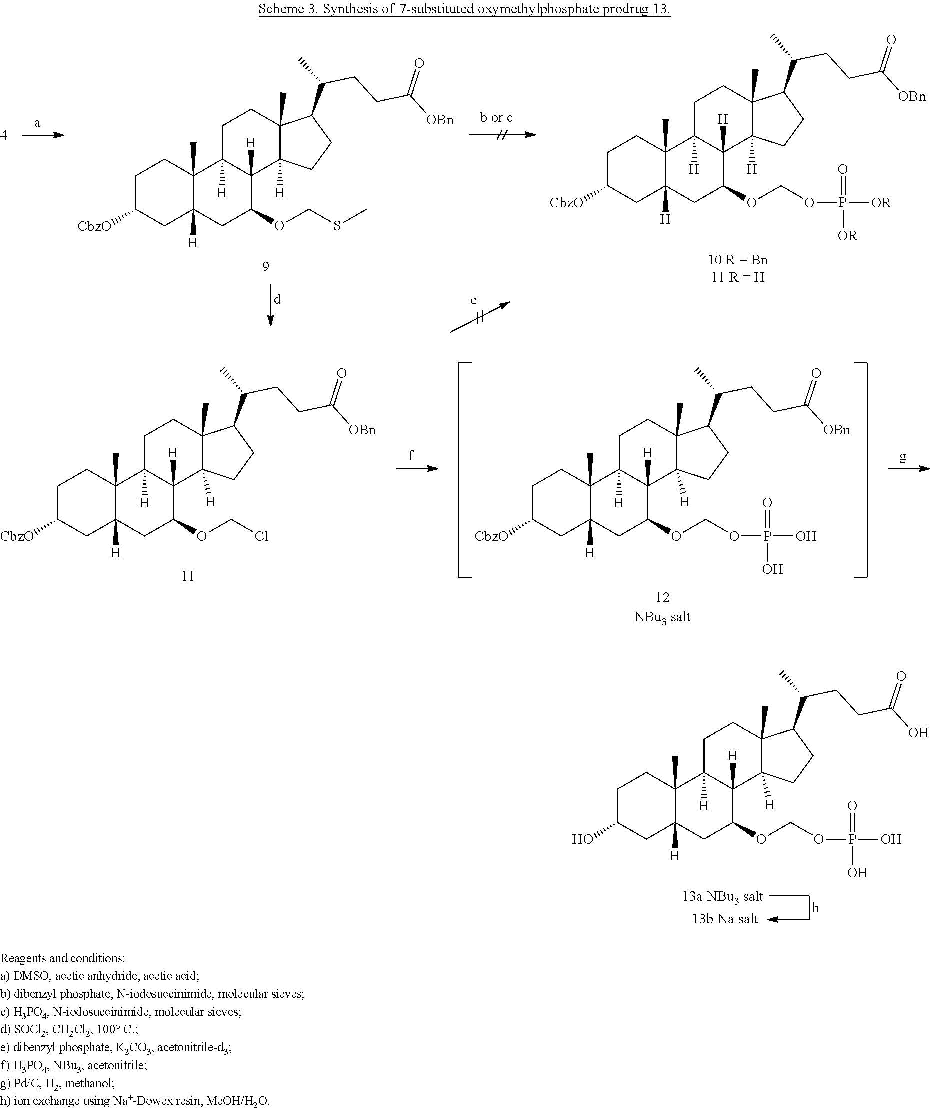

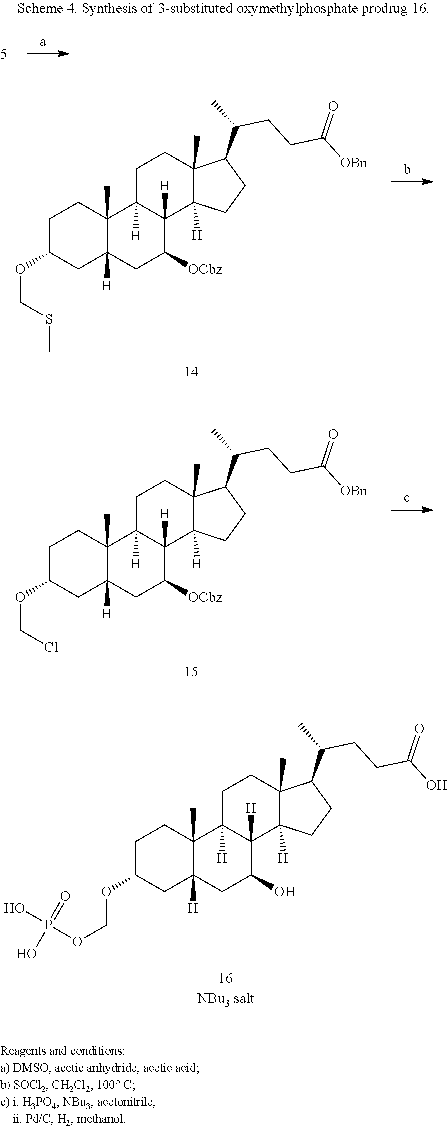

presumably because of its improved solubility in organic solvents. Thus, stirring 11 with a tri(n-butyl)amine salt of phosphate in acetonitrile led to 12, which was then deprotected using hydrogen and Pd/C in methanol. The crude material was purified by Cis column to afford 7-substituted OMP prodrug 13a as an NBu₃ salt. Similarly, 3-substituted OMP prodrug 16 could be obtained from compound 5 using the same sequence of synthetic steps (Scheme 4).

The 3- and 7-substituted oxymethylphosphate prodrugs 16 and 13a were poorly water-soluble as NBu₃ salts. Therefore, compound 13a was converted into a sodium salt 13b by ion-exchange filtration through Dowex resin.^{[28]} The resulting white solid rapidly dissolved in water at all concentrations tested (up to 10 mg/mL). Unfortunately, a significant portion of the material decomposed when left in D₂O solution overnight.

Due to the combination of chemical instability and the relatively difficult synthesis of the 3- and 7-substituted OMP prodrugs, we instead decided to prepare a prodrug where the OMP group is linked to the carboxylic acid of UDCA instead of one of its alcohols. Such a prodrug could potentially be bioactivated in vivo to parent drug both by alkaline phosphatase and by esterases and has the additional advantage that the phosphate moiety is sterically unhindered (relative to the phosphate group in 3 or 7), which may increase the rate of enzymatic activation. We are aware of only one example of such a phosphoryloxymethyl carboxylate (POMC) prodrug in the chemical literature, in a recent patent application by Barnes and coworkers.^{[29]} However, no discussion of the properties of the potential prodrug was presented other than to mention that the material was not obtained cleanly. In addition, Stella and coworkers explored related phosphoryloxymethyloxy carbonyl prodrugs of alcohols, aliphatic amines and aromatic amines, but found their potential utility limited by chemical instability.

We began our synthesis of the POMC prodrug by reacting UDCA with K₂CO₃ and dibenzyl chloromethyl phosphate (17) to afford ester 18 (Scheme 5). Interestingly, this reaction proceeded in higher yield (81% instead of 22%) and at much lower temperature (rt instead of 120° C.) when DMF was used as a solvent instead of acetonitrile. Using DMF instead of acetonitrile also greatly minimized the formation of benzyl ester 1 as a major side product. Next, benzyl deprotection of 18 using hydrogen and Pd/C followed by treatment with sodium carbonate yielded the desired POMC prodrug 19a as a disodium salt. Unfortunately, NMR analysis showed this material to contain a significant amount of impurities and several attempts to synthesize this product cleanly failed. However, we were encouraged by a report from Farquhar and coworkers, who isolated a similar compound that they were using as a chemical intermediate as a dicyclohexylammonium salt.^{[31]} When we replaced sodium carbonate with two equivalents of tris(hydroxymethyl)aminomethane (Tris), we able to cleanly isolate the desired product as a diamine salt (19b). The di-Tris salt of 19 was highly water-soluble, rapidly dissolving at all concentrations tested (up to 20 mg/mL), and stable for extended periods of time when stored in a freezer. However, it showed moderate chemical instability in solution at room temperature (only 36% remained after one week in D₂O solution, Table 1). A diisopropylamine salt (19c) showed similar chemical stability (34% remained after one week at rt in D₂O). However, we noticed that formulations of 19 containing less than two equivalents of amine proved to be significantly more chemically stable in solution. This led us to synthesize the mono-Tris salt of our POMC prodrug, 19d, which was highly water soluble (>20 mg/mL), and decomposed relatively slowly in solution (88% remained after one week at rt in D₂O). The increased aqueous stability of the monoanionic prodrug relative to the dianionic prodrug is similar to that seen with Stella's phosphoryloxymethyloxy carbonyl prodrugs and is consistent with his hypothesis that hydrolysis occurs primarily via an intramolecular general base or intramolecular nucleophilic catalysis mechanism. This hypothesis is further supported by data showing that adding an additional equivalent of Tris to 19b has little effect on its stability in solution (Table 3, entry 19e). The compound also showed similar stability in pH 7.4 tris buffer, with 81% remaining after one day at rt.

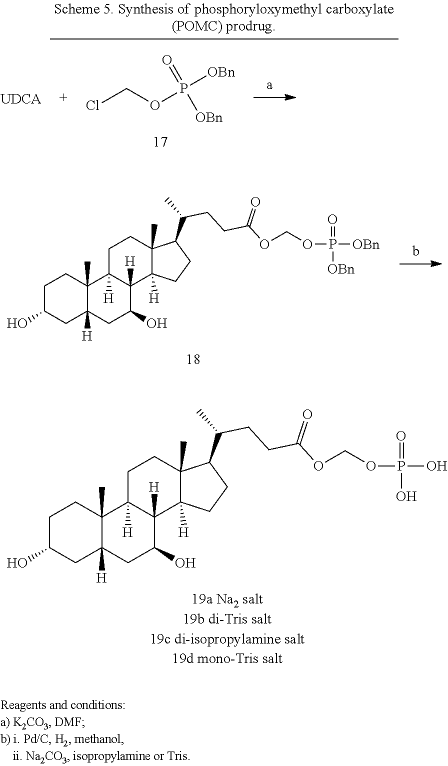

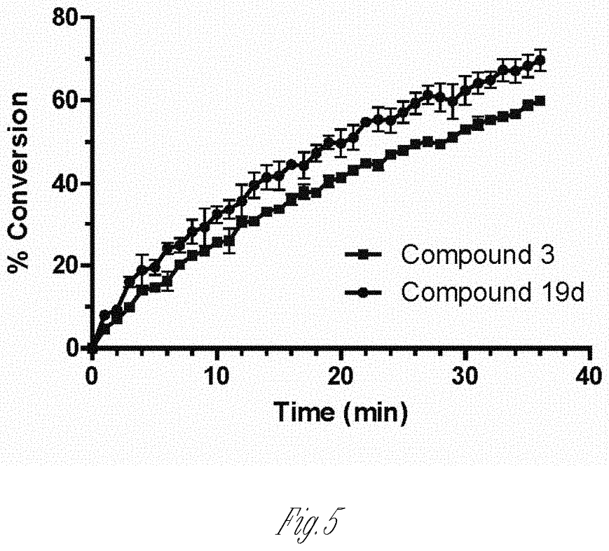

To determine whether the POMC prodrug was indeed activated under in vitro conditions faster than a prodrug where the phosphate moiety is directly linked to an alcohol, we conducted a series of experiments where we monitored the alkaline phosphatase catalyzed activation of prodrugs 19d and 3 by inverse-gated decoupled ³¹P NMR (see Experimental Section for details). As shown in FIG. 5, UDCA is more rapidly released from prodrug 19d under in vitro conditions than prodrug 3.

We have prepared five highly water-soluble prodrugs of the anti-apoptotic bile acid UDCA from three distinct classes: directly linked phosphate esters, oxymethylphosphate (OMP) prodrugs and a novel phosphoryloxymethyl carboxylate (POMC) prodrug. As the OMP prodrugs of UDCA were both difficult to synthesize and chemically unstable, they were not tested in any biological assays. Compound 3, a directly linked phosphate ester, proved to have similar anti-apoptotic potency to UDCA in our in vitro assays, even without prior bioactivation by alkaline phosphatase. Our POMC prodrug compound 19, in contrast, was also highly active in these assays, but required activation by exogenous alkaline phosphatase to have an effect.

The novel POMC prodrug 19 was bioactivated by alkaline phosphatase to UDCA faster than prodrug 3, in which the phosphate ester is directly linked to an alcohol. We were unable to isolate 19 cleanly as a sodium salt, but pure mono and diamine salts of 19 could be readily obtained on a large scale (>5 g) in just two steps from the parent carboxylic acid (UDCA). Diamine salts of 19 were somewhat unstable in solution over long periods of time at ambient temperature, but the mono-Tris salt of 19 decomposed at a much slower rate and was stable for extended periods when stored cold.

Measurement of chemical stability of phosphorylated compounds. 4.0 mg of prodrug (19b, 19c, or 19d) were dissolved in 1.0 mL D₂O. A sealed capillary tube containing phenylphosphonic acid dissolved in D₂O was added as a standard. At time =0, a ¹H NMR spectrum was obtained and the proton signal at □5.51 was integrated (Iₜ₌₀) relative to the aromatic signals from phenylphosphonic acid. After 7 days at rt, a new NMR spectrum was taken and the proton signal at δ 5.51 was integrated again (Iₜ₌₇). The percent starting material remaining was Iₜ₌₇/Iₜ₌₀×100. Each experiment was repeated three times. Chemical stability results obtained either by measuring the disappearance of starting material relative to the internal standard by ³¹P NMR or by using inverse-gated decoupled phosphorus NMR and integrating starting material and product were very similar to the numbers obtained using ¹H NMR. For 19e, 4.0 mg 19b was dissolved in D₂O and an additional equivalent of Tris added (the stoichiometry was confirmed by ¹H NMR) and the experiment conducted as above.
Chemical stability in pH 7.4 tris-buffered saline. 4.0 mg of prodrug 19d was dissolved in 0.9 mL H₂O. To this solution was added 0.1 mL of tris-buffered saline (BM-300 from Boston BioProducts), containing tris (250 mM), KCl (27 mM), and NaCl (1.37 M). A sealed capillary tube containing phenylphosphonic acid dissolved in D₂O was added as a standard. Chemical stability results were obtained by measuring the disappearance of starting material relative to the internal standard by ³¹P NMR. The experiment was repeated three times. The standard deviation was ±3%.
Alkaline phosphatase activation of prodrugs 3 and 19d. Alkaline phosphatase from bovine intestinal mucosa (Sigma-Aldrich, P5521-2KU) was dissolved in 2.0 mL of a 0.100 M sodium glycine buffer containing 1.0 mM ZnCl₂ and 1.0 mM MgCl₂. This stock solution was stored at 4° C. between uses.

Compound 19d (10.0 mg, 0.016 mmol) or compound 3 (8.7 mg, 0.016 mmol) were dissolved in 0.6 mL of a 0.100 M tris glycine buffer solution containing 1.0 mM ZnCl₂ and 1.0 mM MgCl₂. Neither compound showed decomposition by ³¹P NMR when left in this buffer solution for 1 h. 50 μL of the previously prepared AP stock solution was further diluted by addition to 0.950 mL of a 0.100 M tris glycine buffer containing 1.0 mM ZnCl₂ and 1.0 mM MgCl₂. 10.0 μL of this diluted AP solution was added to the prodrug solution by syringe. A series of 42 inverse-gated decoupled ³¹P NMR's were taken (24 scans each, approximately one minute acquisition time). Conversion (%) was determined from the relative integration of the starting material and product peaks, NMR time stamps were used to determine time. Each experiment was repeated three times.

Animal experiments. All experiments involving animals were performed by an Investigator accredited for directing animal experiments (FELASA level C), in conformity with the Public Health Service (PHS) Policy on Humane Care and Use of Laboratory Animals, incorporated in the Institute for Laboratory Animal Research (ILAR) Guide for Care and Use of Laboratory Animals. Experiments received prior approval from the Portuguese National Authority for Animal Health (DGAV).
Cell culture and treatments. Primary rat hepatocytes were isolated from male rats (100-150 g) by collagenase perfusion. Briefly, rats were anesthetized with phenobarbital sodium (100 mg/kg body weight) injected into the peritoneal cavity. After administration of heparin (200 units/kg body weight) in the tail vein, the animals' abdomen was opened and the portal vein exposed and cannulated. The liver was then perfused at 37° C. in situ with a calcium-free

Hanks' Balanced Salt Solution (HBSS) for 10 mM, and then with 0.05% collagenase type IV in calcium-present HBSS for another 10 min. Hepatocyte suspensions were obtained by passing collagenase-digested livers through 125 pm gauze and washing cells in Complete William's E medium (Sigma-Aldrich) supplemented with 26 mM sodium bicarbonate, 23 mM HEPES, 0.01 units/mL insulin, 2 mM L-glutamine, 10 nM dexamethasone, 100 units/mL penicillin, and 10% heat-inactivated fetal bovine serum (Invitrogen). Viable primary rat hepatocytes were enriched by low-speed centrifugation at 200 g for 3 mM. Cell viability was determined by trypan blue exclusion and was typically 80-85%. After isolation, hepatocytes were resuspended in Complete William's E medium and plated on Primaria™ tissue culture dishes (BD Biosciences) at 5×10⁴ cells/cm². Cells were maintained at 37° C. in a humidified atmosphere of 5% CO₂ for 6 h, to allow attachment. Plates were then washed with medium to remove dead cells and incubated in Complete William's E medium supplemented with either 100 μM UDCA, compound 3, compound 19d or no addition (control), in the presence or absence of 3 U/ml of alkaline phosphatase (Invitrogen Corp.) for 12 hours. Cells were then exposed to 1 nmol/L recombinant human TGF-01 (R&D Systems Inc.) for 24 h before processing for cell viability and apoptosis assays.

Cell viability assays. LDH, a stable cytosolic enzyme, is released to cell culture media following cell lysis, and can be used as a marker of cytotoxicity. Briefly, to assess LDH release, supernatants taken from a gentle centrifugation of cell culture media at 250 g, were combined in microplates with lactate (substrate), tetrazolium salt (coloring solution), and NAD (co-factor), previously mixed in equal proportions, following the manufacturer's instructions (Sigma-Aldrich). Multiwell plates were protected from light and incubated for 10 min at room temperature. Finally, absorbance was measured at 490 nm, with 690 nm as reference, using a Bio-Rad model 680-microplate reader (Bio-Rad Laboratories, Hercules, Calif., USA).

To assess cellular viability, the CellTiter-Fluor™ viability assay was used (Promega Corp., Madison, Wis., USA). Briefly, viable cells are measured using a fluorogenic, cell-permeant, peptide substrate (Gly-Phe-AFC), which is cleaved by the live-cell protease activity to generate a fluorescent signal proportional to the number of living cells. Cells were incubated with an equal volume of CellTiter-Fluor™ Reagent for 30 min at 37° C. and resulting fluorescence (380-400 nm_{Ex}/505 nm_{Em}) measured using a GloMax+ Multi Detection System (Promega Corp.).

Apoptosis assays. General caspase-3/7 activity was evaluated using the Caspase-Glo® 3/7 Assay (Promega Corp.). Briefly, the assay provides a proluminescent caspase-3/7 DEVD-aminoluciferin substrate and a proprietary thermostable luciferase in a reagent optimized for caspase-3/7 activity, luciferase activity and cell lysis. Cells were incubated with an equal volume of Caspase-Glo® 3/7Reagent for 30 min at 37° C. and resulting luminescence measured using a GloMax+ Multi Detection System (Promega Corp.).

In addition, Hoechst labeling of cells was used to detect apoptotic nuclei by morphological analysis. Briefly, culture medium was gently removed to prevent detachment of cells. Attached primary rat hepatocytes were fixed with 4% paraformaldehyde in phosphate-buffered saline (PBS), pH 7.4, for 10 min at rt, washed with PBS, incubated with Hoechst dye 33258 (Sigma-Aldrich) at 5 μg/mL in PBS for 5 min, washed with PBS, and mounted using Fluoromount-G™ (SouthernBiotech). Fluorescence was visualized using an Axioskop fluorescence microscope (Carl Zeiss GmbH). Blue-fluorescent nuclei were scored blindly and categorized according to the condensation and staining characteristics of chromatin. Normal nuclei showed non-condensed chromatin disperse over the entire nucleus. Apoptotic nuclei were identified by condensed chromatin, contiguous to the nuclear membrane, as well as by nuclear fragmentation of condensed chromatin. Five random microscopic fields per sample containing approximately 150 nuclei were counted, and mean values expressed as the percentage of apoptotic nuclei.

Statistical analysis. Statistical analysis was performed using GraphPad InStat version 3.00 (GraphPad Software, San Diego, Calif., USA) for the analysis of variance and Bonferroni's multiple comparison tests. Values of p<0.05 were considered significant.
Chemistry. ¹H NMR and ¹³C NMR Spectra were recorded on a Bruker 400 spectrometer. The ¹H NMR data are reported as follows: chemical shift in parts per million downfield of tetramethylsilane (TMS), multiplicity (s=singlet, bs=broad singlet, d=doublet, t=triplet, q=quartet, quint=quintet and m=multiplet), coupling constant (Hz), and integrated value. Coupling constants listed as J_{31P} disappeared when ¹H NMR spectra were taken with ³¹P decoupling. The ¹³C NMR spectra were measured with complete proton decoupling. ³¹P NMR spectra taken for compound characterization were measured with complete proton decoupling and were referenced to 85% phosphoric acid, which was added to the NMR tube in a sealed capillary tube. LC/MS analysis was carried out using a BEH C₁₈ column (2.1 mm×50 mm, 5 um) on a Waters Acquity UPLC system with a Waters ZQ mass detector. Ursodeoxycholic acid was obtained from Sigma-Aldrich. Dibenzyl chloromethyl phosphate was synthesized by the method of Mantyla,^{[34]} but is also commercially available from Sigma-Aldrich.
Ursodeoxycholic acid benzyl ester (1). To a suspension of ursodeoxycholic acid (4.03 g, 10.3 mmol) and K₂CO₃ (4.88 g, 35.3 mmol) in acetonitrile (100 mL) was added benzyl bromide (6.00 mL, 50.5 mmol). The reaction mixture was heated to 80° C. for 3 h, filtered, and concentrated under reduced pressure. Purification by flash chromatography (30% to 100% ethyl acetate/hexanes) on silica gel furnished 4.72 g of white solid (95% yield). ¹H NMR (400 MHz, CD₃OD): 7.39-7.28 (m, 5H), 5.13 and 5.10 (ABq, J_{AB}=12.3 Hz, 2H), 3.56-3.42 (m, 2H), 2.46-2.36 (m, 1H), 2.36-2.25 (m, 1H), 2.08-1.97 (m, 1H), 1.94-1.76 (m, 5H), 1.67-0.98 (m, 18H), 0.97 (s, 3H), 0.94 (d, J=6.4 Hz, 3H), 0.67 (s, 3H). ¹³C NMR (CD₃OD): 12.7, 18.9, 22.4, 23.9, 27.9, 29.6, 31.1, 32.2, 32.3, 35.2, 36.1, 36.6, 38.0, 38.6, 40.7, 41.5, 44.0, 44.5, 44.8, 56.5, 57.5, 67.2, 71.9, 72.1, 129.2, 129.3, 129.6, 137.7, 175.7.
3-(Bis(benzyloxy)phosphoryloxy)-ursodeoxycholic acid benzyl ester (2). To a stirred suspension of ursodeoxycholic acid benzyl ester (1) (1.497 g, 3.10 mmol), 1,2,4-triazole (450 mg, 6.52 mmol), and NaHCO₃ (1.906 g, 22.69 mmol) in 1,2-dichloroethane (30 mL) was added dibenzyl N,N-diethylphosphoramidite (1.00 mL, 3.15 mmol). The reaction mixture was heated overnight to 65° C. After cooling the mixture in an ice bath, THF (12 mL) was added, followed by dropwise addition of 30% H₂O₂ (6 mL). After stirring for 5 min , saturated aqueous Na₂S₂O₃ (30 mL) was added slowly (CAUTION-this is very exothermic). The mixture was diluted with water (200 mL) and extracted with CH₂Cl₂ (2×200 mL). The combined organic layers were dried (MgSO₄), filtered, and concentrated under reduced pressure. Purification by flash chromatography (30% to 100% ethyl acetate/hexanes) on silica gel followed by a second flash chromatography (0 to 10% methanol/CH₂Cl₂) on silica gel furnished 1.1158 g product (48% yield) as a clear colorless oil. ¹H NMR (400 MHz, CDCl₃): 7.43-7.27 (m, 15H), 5.12 and 5.09 (ABq, J_{AB}=12.4 Hz, 2H), 5.07-4.96 (m, 4H), 4.29-4.15 (m, 1H), 3.58-3.44 (m, 1H), 2.46-2.33 (m, 1H), 2.33-2.21 (m, 1H), 2.01-1.93 (m, 1H), 1.93-0.93 (m, 23H), 0.91 (s, 3H), 0.91 (d, J=6.1 Hz, 3H), 0.64 (s, 3H). ¹³C NMR (CDCl₃): 12.1, 18.4, 21.2, 23.2, 26.8, 28.2, 28.6, 31.0, 31.3, 33.9, 34.5, 34.8, 35.2, 36.5, 39.1, 40.1, 42.3, 43.66, 43.73, 54.9, 55.7, 66.1, 69.04, 69.07, 69.10, 69.12, 71.1, 78.6 (d, J_{3IP}=6.0 Hz), 127.88, 127.90, 128.19, 128.25, 128.45, 128.54, 135.98, 136.05, 136.10, 174.0. HRMS calculated for C₄₅H₅₉O₇P+H⁺, 743.4077; observed, 743.4092.
3-(Phosphonatooxy)-ursodeoxycholic acid sodium salt (3). To a solution of compound 2 (2.22 g, 2.99 mmol) in methanol (100 mL) was added 10% Pd/C (291 mg). The reaction mixture was stirred under a balloon filled with hydrogen for 2 h and filtered through celite. Na₂CO₃ (476 mg, 4.49 mmol) dissolved in water (25 mL) was added and solution concentrated under reduced pressure until most of the methanol was removed. The remaining solution was lyophilized to afford 1.627 g of product as a white solid. ¹H NMR (400 MHz, D₂O): 4.04-3.91 (m, 1H), 3.72-3.62 (m, 1H), 2.29-2.17 (m, 1H), 2.17-2.06 (m, 1H), 2.07-1.97 (m, 1H), 1.95-1.00 (m, 23H), 0.96 (s, 3H), 0.95 (d, J=6.0 Hz, 3H), 0.70 (s, 3H). ¹³C NMR (D₂O): 12.1, 18.6, 21.5, 23.3, 27.2, 28.8, 29.0, 33.1, 34.1, 35.2, 35.3, 35.7, 35.8, 37.1, 39.6, 40.4, 42.9, 43.5, 44.0, 55.1, 55.7, 71.9, 75.9 (d, hip=5.0 Hz), 185.5.³¹P NMR (D₂O): 2.51. HRMS calculated for C₂₄H₄₁O₇P+H⁺, 473.2668; observed, 473.2670.
3-(Benzyloxycarbonyloxy)-ursodeoxycholic acid benzyl ester (4) and 7-(benzyloxycarbonyloxy)-ursodeoxycholic acid benzyl ester (5). To a stirred solution of ursodeoxycholic acid benzyl ester 1 (1.465 g, 3.04 mmol) in dry CH₂Cl₂ (50 mL) was added pyridine (0.600 mL, 7.42 mmol) followed by slow addition of benzyl chloroformate (1.00 mL, 7.03 mmol). After stirring for one h, additional pyridine (0.300 mL, 3.71 mmol) and benzyl chloroformate (0.600 mL, 4.22 mmol) were added. After an addition 30 min., the reaction mixture was extracted with 1M HCl (50 mL). The organic layer was dried (Na₂SO₄), filtered, and concentrated under reduced pressure. Purification by flash chromatography (10% to 100% ethyl acetate/hexanes) on silica gel furnished first Compound 4 (0.7769 g, 41% yield) as a slightly yellow foam, followed by Compound 5 (181 mg, 10% yield) as a slightly yellow foam, which was then followed by recovered starting material ursodeoxycholic acid benzyl ester 1 (603.2 mg, 41%) as a white solid. 3-(Benzyloxycarbonyloxy)-ursodeoxycholic acid benzyl ester (4): ¹H NMR (400 MHz, CDCl₃): 7.39-7.30 (m, 10H), 5.14 (s, 2H), 5.12 and 5.09 (ABq, J_{AB}=12.3 Hz, 2H), 4.56 (dddd, J=5, 5, 11, 11 Hz, 1H), 3.60-3.50 (m, 1H), 2.45-2.34 (m, 1H), 2.33-2.22 (m, 1H), 2.02-1.94 (m, 1H), 1.94-0.98 (m, 23H), 0.95 (s, 3H), 0.91 (d, J=6.2 Hz, 3H), 0.65 (s, 3H). ¹³C NMR (CDCl₃): 12.1, 18.3, 21.2, 23.3, 26.4, 26.9, 28.6, 31.0, 31.3, 33.0, 34.1, 34.5, 35.2, 36.6, 39.1, 40.1, 42.2, 43.7, 43.8, 54.9, 55.7, 66.1, 69.3, 71.2, 77.9, 128.18, 128.24, 128.28, 128.46, 128.54, 128.57, 135.4, 136.1, 154.5, 174.0.
7-(Benzyloxycarbonyloxy)-ursodeoxycholic acid benzyl ester (5): ¹H NMR (400 MHz, CDCl₃): 7.39-7.30 (m, 10H), 5.16 and 5.12 (ABq, J_{AB}=12.2 Hz, 2H), 5.12 and 5.10 (ABq, J_{AB}=12.3 Hz, 2H), 4.64 (ddd, J=5, 11, 11 Hz, 1H), 3.63-3.52 (m, 1H), 2.44-2.34 (m, 1H), 2.32-2.22 (m, 1H), 2.01-1.93 (m, 1H), 1.91-0.96 (m, 23H), 0.94 (s, 3H), 0.90 (d, J=6.3 Hz, 3H), 0.62 (s, 3H). ¹³C NMR (CDCl₃): 12.0, 18.3, 21.2, 23.2, 25.6, 28.6, 30.2, 31.0, 31.3, 33.0, 33.9, 34.7, 35.2, 37.1, 39.4, 39.9, 40.0, 42.2, 43.6, 55.0, 55.2, 66.1, 69.3, 71.3, 78.5, 128.11, 128.19, 128.24, 128.39, 128.54, 128.56, 135.6, 136.1, 154.6, 174.0
3-(Benzyloxycarbonyloxy)-7-(bis(benzyloxy)phosphoryloxy)-ursodeoxycholic acid benzyl ester (6). To a stirred suspension of Compound 4 (374 mg, 0.61 mmol), 1,2,4-triazole (89.8 mg, 1.30 mmol), and NaHCO₃ (263 mg, 3.13 mmol) in CH₂Cl₂ was added dibenzyl N,N-diethylphosphoramidite (0.900 mL, 3.00 mmol). The reaction mixture was heated overnight to 40° C. After cooling the mixture in an ice bath, THF (5 mL) was added, followed by dropwise addition of 30% H₂O₂ (3 mL). After stirring for 5 min., saturated aqueous Na₂S₂O₃ (20 mL) was added slowly (CAUTION-this is very exothermic). The mixture was diluted with CH₂Cl₂ and extracted with water (100 mL). The organic layer was dried (Na₂SO₄), filtered, and concentrated under reduced pressure. Purification by flash chromatography (30% ethyl acetate/hexanes) on silica gel furnished 350.1 mg product (66% yield) as a slightly yellow oil. ¹H NMR (400 MHz, CDCl₃): 7.43-7.27 (m, 20H), 5.16 and 5.15 (ABq, J_{AB}=12.4 Hz, 2H), 5.12 and 5.10 (ABq, J_{AB}=12.4 Hz, 2H), 5.05-4.91 (m, 4H), 4.51 (dddd, J=5, 5, 10, 10 Hz, 1H), 4.30-4.17 (m, 1H), 2.45-2.34 (m, 1H), 2.32-2.21 (m, 1H), 1.99-0.99 (m, 24H), 0.93 (s, 3H), 0.90 (d, J =6.1 Hz, 3H), 0.61 (s, 3H). ¹³C NMR (CDCl₃): 12.1, 18.4, 21.2, 23.2, 26.2, 28.4, 31.0, 31.3, 32.7, 33.8, 34.3, 34.4, 35.2, 39.2, 39.8, 41.8, 41.9, 42.0, 43.7, 54.9, 55.0, 66.1, 68.86, 68.92, 69.00, 69.05, 69.4, 77.5, 79.69, 79.75, 127.86, 127.90, 128.17, 128.22, 128.27, 128.40, 128.47, 128.54, 128.59, 135.4, 136.09, 136.11, 136.15, 136.18, 154.5, 174.0. LC/MS calculated for C₅₃H₆₅O₉P+H⁺, 877.4; observed, 877.7.
7-(Phosphonatooxy)-ursodeoxycholic acid sodium salt (7). To a suspension of Compound 6 (1.1984 g, 1.37 mmol) in methanol (200 mL) was added 10% Pd/C (322 mg). The reaction mixture was stirred under a balloon filled with hydrogen for 2 h and filtered through celite. Na₂CO₃ (216.2 mg, 2.04 mmol) dissolved in water (25 mL) was added and solution concentrated under reduced pressure until most of the methanol was removed. The remaining solution was lyophilized to afford 762.7 mg of product as a white solid. ¹H NMR (400 MHz, D₂O): 4.13-3.99 (m, 1H), 3.69-3.55 (m, 1H), 2.30-2.17 (m, 1H), 2.17-2.07 (m, 1H), 2.07-1.92 (m, 3H), 1.92-0.99 (m, 21H), 0.97 (s, 3H), 0.95 (d, J=6.5 Hz, 3H), 0.69 (s, 3H).¹³C NMR (D₂O): 12.1, 18.6, 21.5, 23.4, 27.0, 28.8, 29.6, 33.1, 34.1, 35.0, 35.2, 35.4, 35.9, 36.4, 39.5, 40.2, 42.6, 42.7, 44.0, 55.1, 55.3, 72.0, 76.4 (d, J_{3IP}=5.9 Hz), 185.6. ³¹P NMR (D₂O): 0.93. LC/MS calculated for (C₂₄H₄₁O₇P—H)⁻, 471.3; observed, 471.4.
3-(Benzyloxycarbonyloxy)-7-(methylthiomethoxy)-ursodeoxycholic acid benzyl ester (9). To a solution of Compound 4 (2.71 g, 4.39 mmol) in DMSO (34 mL) was added acetic anhydride (21 mL) followed by acetic acid (34 mL). After stirring at rt for 24 h, the reaction mixture was diluted with water (500 mL) and neutralized with NaHCO₃. The mixture was extracted with ethyl acetate (500 mL). The organic layer was then further extracted with water (5×500 mL), dried (Na₂SO₄), filtered, and concentrated under reduced pressure. Purification by flash chromatography (5% to 30% ethyl acetate/hexanes) on silica gel furnished 1.3966 g of product (47% yield) as a slightly yellow oil. ¹H NMR (400 MHz, CDCl₃): 7.40-7.28 (m, 10H), 5.14 (s, 2H), 5.12 and 5.09 (ABq, J_{AB}=12.4 Hz, 2H), 4.61-4.50 (m, 1H), 4.59 and 4.52 (ABq, J_{AB}=11.2

Hz, 2H), 3.33 (ddd, J=5, 11, 11 Hz, 1H), 2.45-2.35 (m, 1H), 2.32-2.22 (m, 1H), 2.17 (s, 3H), 2.00-1.93 (m, 1H), 1.92-0.97 (m, 23H), 0.95 (s, 3H), 0.90 (d, J=6.2 Hz, 3H), 0.63 (s, 3H). ¹³C NMR (CDCl₃): 12.2, 15.3, 18.4, 21.3, 23.3, 26.3, 26.6, 28.5, 31.0, 31.3, 32.5, 33.0, 34.1, 34.5, 35.2, 39.4, 40.1, 41.5, 42.0, 43.8, 55.0, 55.8, 66.1, 69.4, 73.0, 77.9, 78.1, 128.16, 128.23, 128.28, 128.46, 128.54, 128.57 135.4, 136.2,154.6, 174.1.

3-(Benzyloxycarbonyloxy)-7-(chloromethoxy)-ursodeoxycholic acid benzyl ester (11). To a solution of Compound 9 (847 mg, 1.25 mmol) in dry CH₂Cl₂ (20 mL) was added 2M SOCl₂ in CH₂Cl₂ (1.9 mL, 3.8 mmol). The reaction mixture was heated in a microwave to 100° C. for 30 min. and then concentrated under reduced pressure. A ¹H NMR spectrum of the crude material in CDCl₃ showed a new set of doublets 5 5.56 and 5.47 (J=5.4 Hz, 1H each)^{[35]} and the disappearance of the AB pattern at 5 4.59 and 4.52 as well as the SMe peak which had been at 5 2.17 in the ¹H NMR spectrum of Compound 9. The crude material was used without further purification in the next reaction.
7-(Phosphonooxymethoxy)-ursodeoxycholic acid tributylamine salt (13a). To a suspension of H₃PO₄ (586 mg, 5.98 mmol) and 4 A molecular sieves (2.023 g) in acetonitrile (40 mL) was added Bu₃N (5.4 mL, 22.7 mmol). The mixture was stirred overnight and then added to a flask containing crude 11. After stirring for 24 h, the mixture was filtered through celite and concentrated under reduced pressure. The residue was dissolved in methanol (50 mL) and concentrated under reduced pressure again. Next, the residue was dissolved in methanol (50 mL), 10% Pd/C (2.369 g) added, and the reaction mixture stirred under a balloon filled with hydrogen for 2 h and then filtered through celite. Additional 10% Pd/C (2.14 g) was added and the reaction mixture stirred under a balloon filled with hydrogen for 72 h. The reaction mixture was filtered through celite and concentrated under reduced pressure. The resulting residue purified by chromatography (5% acetonitrile/water to 100% acetonitrile, C₁₈ column) to yield 116.7 mg white solid after lyophilization. There are approximately 1.4 equivalents of NBu₃ present for every equivalent of bile acid based on ¹H NMR analysis (comparison of the integration of the methyl peak at 5 0.70 to the multiplet at 5 3.12-3.02). ¹H NMR (400 MHz, CD₃OD): 5.18 (dd, J=6 Hz, J_{31P}=6 Hz, 1H), 4.99 (dd, J=6 Hz, J_{31P}=8 Hz, 1H), 3.66-3.55 (m, 1H), 3.53-3.42 (m, 1H), 3.13-3.02 (m, 8.2H), 2.35-2.24 (m, 1H), 2.20-2.10 (m, 1H), 2.08-1.98 (m, 1H), 1.94-0.90 (m, 57H), 0.70 (s, 3H). LC/MS calculated for (C₂₅H₄₃O₈P—H)⁻, 501.3; observed, 501.3.
7-(Phosphonooxymethoxy)-ursodeoxycholic acid sodium salt (13b). A 1 cm wide column was filled with 12 cm of DOWEX 50W2 (50-100 mesh, strongly acidic) ion exchange resin.^{[28]} The column was prepared by sequentially washing with 1:1 methanol/water, 1M aqueous NaHCO₃ (lots of gas evolution), water, and then finally 1:1 methanol/water. Compound 13a (115 mg) was dissolved in 1:1 methanol/water and loaded onto the column, which was eluted with 1:1 methanol/water. The product containing fractions were lyophilized to furnish the product as a white solid (76.4 mg). ¹H NMR (400 MHz, D₂O ): 5.18 (dd, J=5.7 Hz, J_{31P}=6.8 Hz, 1H), 4.99 (dd, J=5.7 Hz, J_{31P}=9.4 Hz, 1H), 3.74-3.56 (m, 2H), 2.40-2.28 (m, 1H), 2.27-2.14 (m, 1H), 2.08 (m, 24H), 1.00-0.92 (m, 6H), 0.70 (s, 3H).
3-(Methylthiomethoxy)-7-(benzyloxycarbonyloxy)-ursodeoxycholic acid benzyl ester (14). To a solution of Compound 5 (1.113 g, 1.80 mmol) in DMSO (17 mL) was added acetic anhydride (10.5 mL) followed by acetic acid (17 mL). After stirring at rt for 24 hours, the reaction mixture was diluted with water (500 mL) and neutralized with NaHCO₃. The mixture was extracted with ethyl acetate (500 mL). The organic layer was then further extracted with water (5×500 mL), dried (Na₂SO₄), filtered, and concentrated under reduced pressure. Purification by flash chromatography (5% to 50% ethyl acetate/hexanes) on silica gel furnished 364 mg of product (30% yield) as a slightly yellow oil. ¹H NMR (400 MHz, CDCl₃): 7.41-7.28 (m, 10H), 5.16 and 5.12 (ABq, J_{AB}=12.0 Hz, 2H), 5.12 and 5.10 (ABq, J_{AB}=12.4 Hz, 2H), 4.67-4.58 (m, 1H), 4.65 (s, 2H), 3.57 (dddd, J=5, 5, 10, 10 Hz, 1H), 2.44-2.34 (m, 1H), 2.31-2.22 (m, 1H), 2.15 (s, 3H), 2.00-1.93 (m, 1H), 1.92-0.95 (m, 23H), 0.94 (s, 3H), 0.89 (d, J=6.3 Hz, 3H), 0.62 (s, 3H). ¹³C NMR (CDCl₃): 12.2, 13.7, 18.3, 21.2, 23.2, 25.6, 26.9, 28.4, 31.0, 31.3, 33.1, 33.4, 34.2, 34.7, 35.2, 39.2, 39.9, 40.0, 42.2, 43.6, 55.0, 55.2, 66.1, 69.3, 72.0, 75.2, 78.5, 128.10, 128.18, 128.24, 128.39, 128.64, 135.6, 136.1, 154.6, 174.0.
3-(Chloromethoxy)-7-(benzyloxycarbonyloxy)-ursodeoxycholic acid benzyl ester (15). To a solution of Compound 14 (360 mg, 0.53 mmol) in dry CH₂Cl₂ (20 mL) was added 2M SOCl₂ in CH₂Cl₂ (0.8 mL, 1.6 mmol). The reaction mixture was heated in a microwave to 100° C. for 30 min. and then concentrated under reduced pressure. A ¹H NMR spectrum of the crude material in CDCl₃ showed a new AB pattern at 5 5.55 and 5.54 (J=5.4 Hz, 2H total) and the disappearance of the singlet at 5 4.65 as well as the SMe peak which had been at 5 2.15 in the ¹H NMR spectrum of Compound 14. The crude material was used without further purification in the next reaction.
3-(Phosphonooxymethoxy)-ursodeoxycholic acid tributylamine salt (16). To a suspension of H₃PO₄ (248 mg, 2.53 mmol) and 4 A molecular sieves (0.760 g) in acetonitrile (15 mL) was added Bu₃N (2.3 mL, 9.68 mmol). The mixture was stirred overnight and then added to a flask containing the crude product of the previous reaction. After stirring for 72 h, the mixture was filtered and concentrated under reduced pressure. The residue was dissolved in methanol (25 mL) and concentrated under reduced pressure again. Next, the residue was dissolved in methanol (40 mL), 10% Pd/C (656 mg) added, and the reaction mixture stirred under a balloon filled with hydrogen for 2 h and then filtered through celite. A crude NMR of an aliquot showed no reaction. Additional 10% Pd/C (744 mg) was added and the reaction mixture stirred under a balloon filled with hydrogen for 2 h and filtered through celite. A crude NMR of an aliquot again showed no reaction. Additional 10% Pd/C (1901 mg) was added and the reaction mixture stirred under a balloon filled with hydrogen overnight, filtered through celite and concentrated under reduced pressure. The resulting residue purified by chromatography (5% acetonitrile/water to 100% acetonitrile, C₁₈ column) to yield 80.7 mg white solid after lyophilization. There are approximately 1.7 equivalents of NBu₃ present for every equivalent of bile acid based on ¹H NMR analysis (comparison of the integration of the methyl peak at δ 0.71 to the multiplet at 5 3.12-3.02). ¹H NMR (400 MHz, CD₃OD): 5.08 (d, J_{31P}=8.4 Hz, 2H), 3.75-3.63 (m, 1H), 3.54-3.43 (m, 1H), 3.12-2.98 (m, 10H), 2.34-2.23 (m, 1H), 2.19-2.09 (m, 1H), 2.08-20 (m, 1H), 1.94-0.90 (m, 68H), 0.71 (s, 3H). LC/MS calculated for (C₂₅H₄₃O₈P—H)⁻, 501.3; observed, 501.3.
Ursodeoxycholic acid (bis(benzyloxy)phosphoryloxy)methyl ester 18. To a suspension of ursodeoxycholic acid (1.46 g, 3.72 mmol) and K₂CO₃ (984 mg, 7.12 mmol) in DMF (10 mL) was added dibenzyl chloromethyl phosphate (1.23 g, 3.76 mmol). The mixture was stirred overnight, diluted with water (250 mL), and extracted with ethyl acetate (3×250 mL) and CH₂Cl₂ (1×250 mL). The combined organic layers were dried (MgSO₄), filtered, and concentrated under reduced pressure. Purification by flash chromatography (40% to 100% ethyl acetate/hexanes) on silica gel furnished 2.05 g of clear, colorless foamy oil (81% yield). ¹H NMR (400 MHz, CD₃OD): 7.42-7.38 (m, 10H), 5.64 (d, =13.8 Hz, 2H), 5.10 (d, J_{31P}=8.3 Hz, 4H), 3.58-3.44 (m, 2H), 2.41-2.31 (m, 1H), 2.29-2.18 (m, 1H), 2.08-1.98 (m, 1H), 1.96-1.72 (m, 5H), 1.70-1.00 (m, 18H), 0.99 (s, 3H), 0.93 (d, J=6.5 Hz, 3H), 0.71 (s, 3H). ¹³C NMR (CD₃OD): 12.7, 18.9, 22.4, 23.9, 27.9, 29.6, 31.1, 31.6, 31.8, 35.2, 36.1, 36.5, 38.0, 38.6, 40.7, 41.5, 44.0, 44.5, 44.8, 56.4, 57.5, 71.1 (d, J_{31P}=5.9 Hz), 71.9, 72.1, 83.9 (d, J_{31P}=5.7 Hz), 129.2, 129.7, 129.8, 136.9, 137.0, 173.8.³¹P NMR (CD₃OD): -1.59. HRMS calculated for C₃₉H₅₅O₈P+H⁺, 683.3713; observed, 683.3735.
General Procedure for the Synthesis of Salts of Ursodeoxycholic Acid phosphonooxymethoxy ester (19). To a solution of 18 in methanol was added 10% Pd/C. The reaction mixture was stirred under a balloon filled with hydrogen for 45 min and filtered through celite Amine was added (1 or 2 equivalents) and the solution concentrated under reduced pressure. Data for mono-Tris Salt (19(1)¹H NMR (400 MHz, D₂O): 5.51 (d, J_{31P}=12.8 Hz, 2H), 3.74 (s, 6H), 3.60-3.54 (m, 2H), 2.58-2.46 (m, 1H), 2.44-2.32 (m, 1H), 2.09-1.98 (m, 1H), 1.96-1.74 (m, 5H), 1.72-1.02 (m, 18H), 1.01-0.95 (m, 6H), 0.72 (s, 3H). ¹³C NMR (D₂O): 12.6, 19.0, 22.0, 23.9, 27.2, 29.0, 30.2, 31.1, 31.4, 34.4, 35.4, 35.7, 36.6, 37.3, 39.9, 40.8, 42.8, 43.7, 44.1, 55.3, 56.1, 60.0, 62.1, 71.6, 71.7, 83.6, 176.4. ³¹P NMR (D₂O): -0.30. HRMS calculated for (C₂₅H₄₃O₈P—H)⁻, 501.2617; observed, 501.2585.

Patients with neurodegenerative diseases such as Parkinson's disease and Alzheimer's disease; Huntington's disease; multiple sclerosis; amyotrophic lateral sclerosis; cerebellar ataxia; lysosomal storage disorders; can greatly benefit from the neuroprotective properties of bile acids either alone or in combination with pro-drugs.

Along these lines, antioxidants such as the bile acids, ursodeoxycholic acid (UDCA) and tauroursodeoxycholic acid (TUDCA), and their analogues and derivatives are novel agents for the reduction of risk of neurodegenerative diseases. UDCA is a hydrophilic tertiary bile acid that is normally produced endogenously in the liver. Although hydrophilic bile acids, such as glycochenodeoxycholic acid and taurochenodeoxycholic acid, are toxic and induce programmed cell death, UDCA and TUDCA are non-toxic. TUDCA can not only prevent hepatic cell death but also block oxygen radical production and programmed cell death in non-hepatic cells including neuronal cells.

In one embodiment, phosphorylated bile acids and all derivatives and precursors thereof with or without pro-drugs protect neurons and brain tissue from degeneration or toxicity.

In one embodiment, phosphorylated bile acids and all derivatives and precursors thereof with or without pro-drugs protect neurons and brain tissue from apoptosis.

In one embodiment, phosphorylated bile acids and all derivatives and precursors thereof with or without pro-drugs protect neurons and brain tissue from reactive oxidative damage.

In one embodiment, phosphorylated bile acids and all derivatives and precursors thereof with or without pro-drugs protect neurons and brain tissue from mitochondrial dysfunction or destruction.

In one embodiment, phosphorylated bile acids and all derivatives and precursors thereof with or without pro-drugs prevents or abolishes apoptosis in neurons and brain tissues.

In another embodiment of this invention, phosphorylated bile acids and all derivatives and precursors thereof can be conjugated to any anti-neurodegenerative pro-drug molecules involved in modulating neuronal apoptosis.

In another embodiment of this invention, phosphorylated bile acids and all derivatives and precursors thereof can be conjugated to pro-drugs of DA neurons such as L-DOPA and any analog of L-DOPA.

In another embodiment of this invention, phosphorylated bile acids and all derivatives and precursors thereof are conjugated to glutamate receptor antagonists.

In another embodiment of this invention, phosphorylated bile acids and all derivatives and precursors thereof are conjugated to antioxidants.

In another embodiment of this invention, phosphorylated bile acids and all derivatives and precursors thereof can be combined, without conjugation, to any anti-neurodegenerative pro-drug molecules involved in modulating neuronal apoptosis.

In another embodiment of this invention, phosphorylated bile acids and all derivatives and precursors thereof can be combined, without conjugation, to pro-drugs of DA neurons such as L-DOPA and any analog of L-DOPA.

In another embodiment of this invention, phosphorylated bile acids and all derivatives and precursors thereof are combined, without conjugation, to glutamate receptor antagonists.

In another embodiment of this invention, phosphorylated bile acids and all derivatives and precursors thereof are combined, without conjugation, to antioxidants.

The term “effective amount” as used herein includes useful dosage levels of the compound of the present invention that will be effective to prevent or mitigate or completely cure the patients of any neurodegenerative disease. Useful dosages of the desired compound described herein can be determined by comparing its in vitro activity and its in vivo activity in animal models. Methods for extrapolation of effective dosages in mice, and other animals, to humans are known in the art.

It will be understood, however, that the specific “effective amount” for any particular subject will depend upon a variety of factors including the activity of the specific compound employed; the age, body weight, general health, sex, diet, time of administration, route of administration, rate of excretion, drug combination, and the severity of the medical condition for the subject being treated.

The phosphorylated bile acids and their derivatives or precursors with or without pro-drugs are used in amounts effective to treat Parkinson's disease or any other neurodegenerative disease by either or both prophylactic or therapeutic treatments. Treatment involves prevention of onset or retardation or complete reversal of any or all symptoms or pharmacological or physiological or neurological or biochemical indications associated with Parkinson's disease or other neurodegenerative disease. Treatment can begin wither with the earliest detectable symptoms or established symptoms of Parkinson's disease or other neurodegenerative disease.

The “effective” amount of the compound thereof is the dosage that will prevent or retard or completely abolish any or all pathophysiological features associated with various stages (late or end) Parkinson's disease (sporadic or familial) or other neurodegenerative disease.

The phosphorylated bile acids and their derivatives or precursors with or without pro-drugs can be combined with a formulation that includes a suitable carrier. Preferably, the compounds utilized in the formulation are of pharmaceutical grade. This formulation can be administered to the patent, which includes any mammal, in various ways which are, but not limited to, oral, intravenous, intramuscular, nasal, or parental (including, and not limited to, subcutaneous, intramuscular, intraperitoneal, intravenous, intrathecal, intraventricular, direct injection into the brain or spinal tissue).

Formulations may be presented to the patient may be prepared by any of the methods in the realm of the art of pharmacy. These formulations are prepared by mixing the biologically-active bile acid and its derivative or precursor with or without pro-drugs into association with compounds that comprise the carrier. The carrier can be liquid, granulate, solid (coarse or finely broken), liposomes (including liposomes prepared in combination with any non-lipid small or large molecule), or any combination thereof.

The formulation in the current invention can be furnished in distinct units including, but not limited to, tablets, capsules, caplets, lozenges, wafers, troches with each unit containing specific amounts of the active molecule for treating Parkinson's or other neurodegenerative disease. The active molecule can be incorporated either in a powder, encapsulated in liposomes, in granular form, in a solution, in a suspension, in a syrup, in any emulsified form, a drought or an elixir.

Tablets, capsules, caplets, pills, troches, etc. that contain the biologically-active bile acid and its derivatives or precursors with or without pro-drugs can contain binder (including, but not limited to, corn starch, gelatin, acacia, bum tragacanth), an excipient agent (including but not limited to dicalcium phosphate), a disintegrating agent (including but not limited to corn starch, potato starch, alginic acid) a lubricant (including but not limited to magnesium stearate), a sweetening agent (including but not limited to sucrose, fructose, lactose, aspartame), a natural or artificial flavoring agent. A capsule may additionally contain a liquid carrier. Formulations can be of quick or sustained or extended-release type.

Syrups or elixirs can contain one or several sweetening agents, preservatives, crystallization-retarding agents, solubility-enhancing agents, etc.

Any or all formulations containing the biologically-active bile acids and their precursors or derivatives with or without pro-drugs can be included into the food (liquid or solid or any combination thereof) of the patient. This inclusion can either be an additive or supplement or similar or a combination thereof.

Parenteral formulations are sterile preparations of the desired biologically-active bile acid and its precursor or derivative with or without pro-drugs can be aqueous solutions, dispersions of sterile powders, etc., that are isotonic with the blood physiology of the patient. Examples of isotonic agents include, but are not limited to, sugars, buffers (example saline), or any salts.

Formulations for nasal spray are sterile aqueous solutions containing the biologically-active bile acid and its precursors or derivatives with or without pro-drugsalong with preservatives and isotonic agents. The sterile formulations are compatible with the nasal mucous membranes.

The formulation can also include a dermal patch containing the appropriate sterile formulation with the active agent. The formulation would release the active agent into the blood stream either in sustained or extended or accelerated or decelerated manner

The formulation can also consist of a combination of compounds, in any of the afore mentioned formulations designed to traverse the blood-brain barrier.

### EXAMPLES

In the following examples, the role of biologically-active bile acid in the protection of neurons from destruction or dysfunction is described. In a dose-dependent manner, UDCA prevented sodium nitroprusside (SNP)-induced cytotoxicity in human dopaminergic SH-SYSY cells. UDCA effectively attenuated the production of total reactive oxygen species (ROS), peroxynitrite (ONOO⁻) and nitric oxide (NO), and markedly inhibited the mitochondrial membrane potential (MMP) loss and intracellular reduced glutathione (GSH) depletion.

In another example, SNP-induced programmed cell death or apoptotic events, such as nuclear fragmentation, caspase-3/7 and -9 activation, Bcl-2/Bax ratio decrease, and cytochrome c release, were significantly attenuated by UDCA.

In another example, the selective inhibitor of phosphatidylinositol-3-kinase (P13K), LY294002, and Akt/PKB inhibitor, triciribine, reversed the preventive effects of UDCA on the SNP-induced cytotoxicity and Bax translocation. These results indicate that UDCA can protect SH-SYSY cells under programmed cell death process by regulating P13K-Ak1/PKB pathways.

### Methods

### Cell Culture and Treatments

Human dopaminergic neuronal cell line, SH-SYSY, was cultured in DMEM/F12 medium supplemented with 10% FBS (v/v), penicillin (100 U/ml)-streptomycin (100 μg/ml) in 5% CO₂ at 37° C. SH-SYSY cells were cultured at a seeding density of 3×10⁵ cells/ml. Usually, the culture medium was changed to DMEM/F12 medium with 0.5% FBS before any treatment to reduce the serum effect. In order to prevent the direct interaction between the treated chemicals, the culture medium was changed to fresh low-serum medium at the ent of pretreatment. UDCA was dissolved in ethanol as a 100× stock solution and diluted to the desired final concentrations. To estimate cell viability, 3-(4,5-dimetnylthiazol-2-yl)-2,5-diphenyltetrazolium bromide (MTT) reduction assay was performed. After cells were treated ad culture medium was removed, MTT solution (50 μg, 1 mg/ml in phosphate buffered saline, PBS) was added to each well in 96-well plate and incubated for 4 h at 37° C. The medium was carefully removed, 100 μl DMSO added to each well, and the plate agitated on an orbital shaker for 15 mM to dissolve the formazan. The absorbance was measured at 540 nm using a microplate reader (SpectraMax M2, Molecular Devices).

### Nuclear Staining for Detecting Apoptosis and Necrosis

For the fluorescent detection of apoptotic and necrotic cells, nuclear staining with Hoechst dye 33342 and propidium iodide (PI) was performed. SH-SYSY cells were exposed to SNP (1 mM) for 24 h with or without pretreatment with UDCA or YS. After fixation with 1% paraformaldehyde (PBS) for 30 min at room temperature, cells were washed with PBS and then stained with Hoechst 33342 (10 μM) for 10 mM Cells were washed with PBS and further stained with PI (10 μM) for 10 min. Stained cells were washed with PBS and observed under a fluorescent microscopy. The apoptotic cells were determined as bright condensed and fragmented nuclei. PI positive cells stained with pink to red color were counted as necrotic cells.

### Analysis of Caspase Activity

Caspase-3/7 and caspase-9 activities were measured using the fluorogenic substrates. The assay was performed according to the manufacturer's protocol (Sensolyte Homogenous AMC Caspase Assay Kit, Anaspec Inc.). Briefly, cells were seeded at 3×10⁴ cells/well in 96-well black wall and clear bottom culture plates. After 1 day, cells were pretreated for 1 h with UDCA (50, 100, 200 μM) or YS (100, 200 μM) then treated with SNP (1 mM) for 12 h. The fluorogenic peptide substrates Ac-DEVD-AMC and Ac-LEHD-AMC were used for caspase-3/7 and caspase-9, respectively. The reaction buffer containing 40 mM DTT and 100 μM substrate peptide was added into each well (50 μl of reaction buffer/well) and mixed completely by shaking and then incubated for 1 h. Fluorescende was read at 354 excitation and 442 emission on a fluorescence microplate reader (SpectraMax M2, Molecular Devices).

### Detection of Total ROS, ONOO⁻, and NO Levels

The production of total ROS was measured using 2′,7′-dichlorodihydrofluorescein diacetate (H₂DCFDA, Sigma-Aldrich) and the formation of peroxynitrite ONOO⁻) was determined using dihydrorhodamine 123 (DHR 123, Molecular Probes). SH-SYSY cells were treated with SNP (1 mM) with or without various concentrations of UDCA or YS for 12 h. After washing with Hank's balanced salt solution (HBSS), cells were incubated with 20 μM H₂DCFDA or 50 μM DHR at 37° C. for 30 mM, and then rinsed with HBSS. The fluorescence intensity was measured using an automatic fluorescence microplate reader (SpectraMax M2, Molecular Devices) at an excitation wavelength of 485 nm and an emission of 535 nm. The values were expressed as a percentage of fluorescence intensity to the untreated control group. The production of NO was determined by measuring nitrite, a stable oxidation product of NO in the culture medium. After treatment of SNP (1 nM) with or without various concentrations of UDCA or YS for 24 h, cell culture medium was mixed with an equal volume of Griess reagent (Sigma-Aldrich). After a 10-min reaction, the absorbance at 550 nm was measured in a microplate reader (VersaMax, Molecular Devices). Sodium nitrite (NaNO₂) was used as a standard to calculate nitrite concentrate and the values were expressed in micromoles.

### Measurement of Mitochondrial Membrane Potential (MMP)

MMP (ΔΨm) was measured using the mitochondria-specific lipophilic cationic fluorescent dye 5,5′,6,6′-tetrachloro-1,1′,3,3′-tetraethybenzimidazolocarbocyanine iodide (JC-1; Anaspec Inc.). JC-1 preferentially accumulates in mitochondria as red aggregates in normal conditions but it exists as green monomers in the cytosol when MMP collapsed during apoptosis. The ratio of red/green fluorescence correlates with MMP. SH-SYSY cells were pretreated with various concentrations of UDCA or YS for 1 h and then treated with 1 mM SNP for additional 12 h. Next, 5 μg/ml JC-1 was added and incubated at 37° C. for 15 mM in dark. After wash three times with PBS, MMP was measured at 535/590 nm (Ex/Em) for red fluorescence and 485/535 (Ex/Em) for green fluorescence using a fluorescence multimode microplate reader (Infinite 200; Tecan). Results were calculated as the ratio of red-to-green fluorescence and the values were expressed as the percentage over control.

### Measurement of Cellular Reduced Glutathione (GSH) Content

The intracellular GSH levels were analyzed using the fluorescent dye monochlorobimane (MCB, Sigma-Aldrich). Briefly, following treatments, SH-SYSY cells in black 96-well culture plates were washed with HNSS and then incubated with 40 μM MCB for 20 mM in dark. After washing twice with HBSS, fluorescence intensity was determined at 355/460 nm (Ex/Em) in a fluorescence microplate reader (SpectraMax M2, Molecular Devices). GSH content was determined from a standard curve constructed using known amounts of glutathione (Sigma-Aldrich). Values were expressed as a relative content of untreated group.

### Immunoblot Analysis

SH-SYSY cells were pretreated for 1 h with UDCA (200 μM) and then treated with SNP (1 mM) for fixed time according to our pretests (12 h for the analysis of Bcl-2, Bax, and cytochrome c). Whole cell proteins were extracted using RIPA buffer (PBS, 1% NP-40, 0.5% Na deoxycholate, 0.1% SDS, 0.1 mg/ml PMSF, 30 mg/ml aprotinin, 1 mM Na₃VO₄). Cells were washed twice with PBS, lysed with RIPA buffer for 30 mM on ice, and then centrifuged at 14,000×g for 10 mM at 4° C. The supernatants were used as the total cell lysates. In some experiments, mitochondrial fraction was prepared from SH-SySY cells using mitochondrial/cytosolic fraction kit (Biovision, Inc., Mountain View) according to the manufacturer's protocol. Protein concentration was determined by BCA protein assay kit (BioRad, Hercules, Calif.) using bovine serum albumin as a standard. Protein samples (40 μg) were separated on a 10-15% SDS-polyacrylamide gel and transferred onto PVDF membrane. The membrane was flocked in fresh blocking buffer (5% nonfat dry milk in Tris-buffered saline, pH 7.4, and containing 0.1% Tween 20) for 2 h at room temperature and rinsed in TBST buffer (0.1% Tween 20 in Tris-buffered saline, pH 7.4). The membrane was incubated at 4° C. with the following primary antibodies at dilutions of 1/1000: Bax, cytochrome c, Cox-4 or 1/4000: Bcl-2, actin. After three times washing with TBST buffer, membranes were incubated with horse radish peroxidase (HRP)-conjugated secondary antibodies (1:2000 dilutions) for 2 h at room temperature. Subsequently, the membrane was washed in TBST and the immunoreactive bands were detected by ECL chemiluminescence kit (GE Healthcare, USA). Protein bands were quantified by densitometric analysis.

### Statistical Analysis

All experiments were performed at least three times, and results were expressed as the mean ±SEM. The data were analyzed using the SPSS 12.0 software package (SPSS Inc., Chicago, Ill.). Differences were analyzed using one-way factorial analysis of variance (ANOVA), and the Duncan's post hoc test.

### Results

Protective Effect of UDCA and YS against SNP-induced Neurotoxicity

Initial studies were performed to examine the cytotoxic response of SH-SYSY cells to various concentrations (100 μM-2 mM) of SNP. The loss of viability occurred by SNP in a dose-dependent manner, and 1 mM SNP induced approximately 56% cell loss after 24 hr of treatment. Thus, we did subsequent experiments using 1 mM SNP. Treatment with UDCA alone or YS alone for 24 h at doses of 50-200 μM showed no obvious change in the viability compared with the control group. To investigate the effect of UDCA and YS on SNP-induced human dopaminergic cell death, SH-SYSY cells were pretreated with 50-200 μM UDCA or 100-200 mM YS for 1 h, followed by 1 mM SNP treatment for 24 h. SNP-induced loss of cell viability was significantly attenuated by UDCA or YS pretreatment dose-dependently.

Although SNP acts as a NO donor, the molecular structure of SNP shows a complex of NO with ferrous ion and five cyanides. Therefore, SNP not only produces NO but also generates cyanides and free iron. To distinguish the role of NO, cyanides, and free iron in the SNP-induced dopaminergic cell death, SH-SYSY cells were treated with potassium ferricyanide or sodium cyanide. However, treatment with potassium ferricyanide (0.5, 1 mM) or sodium cyanide (0.5 or 1 mM) did not change the cell viability obviously. Also, to confirm a causative role of NO moiety in SNP, we treated SH-SYSY cells with the 50 day light exposed SNP (SNP_{EXP}), which corresponds to its NO-exhausted SNP. SNP_{EXP} did not effect the cell viability of SH-SYSY cells. Thus, we can speculate that NO may be a cytotoxic mediator involved in SNP-induced dopaminergic cell death.

UDCA and YS ameliorated SNP-induced apoptosis and caspase activation

We investigated the effect of UDCA and SNP-induced programmed cell death characteristics, such as nuclear morphology changes, caspase-3/7 activation and caspase-9 activation in SH-SYSY cells. A significant proportion of SNP-induced cell death was apoptotic, based on Hoechst 33342-stained nuclear changes in morphology and PI staining. We observed a significant increase in condensed, fragmented nuclei after 24 h treatment with SNP (1 mM). However, a low percentage of nuclei were stained red by the necrotic marker dye PI. The number of those hallmarks of apoptotic or necrotic nuclei was similar to untreated control cells and both UDCA and YS treated cells. Moreover, we found that both UDCA and YS effectively inhibited SNP-mediated apoptotic nuclear damages. As quantified in, although SNP increased the apoptotic rate to 30.59+3.38%, UDCA or YS pretreatment prior to SNP treatment caused a statistically significant reduced apoptotic rate (8.45+2.01% and 11.67+1.75%, respectively).

Next, we examined caspase-3/7 and caspase-9 activity as another marker of programmed cell death. The exposure of SH-SYSY cells to 1 mM SNP for 12 h increased caspase-3/7 and -9 activities by 2.43 and 4.21-fold respectively. Either UDCA (50-200 μM) or YS (100-200 μM) pretreatment strongly attenuated the effects of SNP on caspase-3/7 and caspase-9 activity. These results suggest that the protective effects of UDCA and YS are mediated by anti-apoptotic pathway.

### UDCA and YS Inhibited SNP-induced NO, ONOO⁻, and Total ROS Production in SH-SY5Y Cells

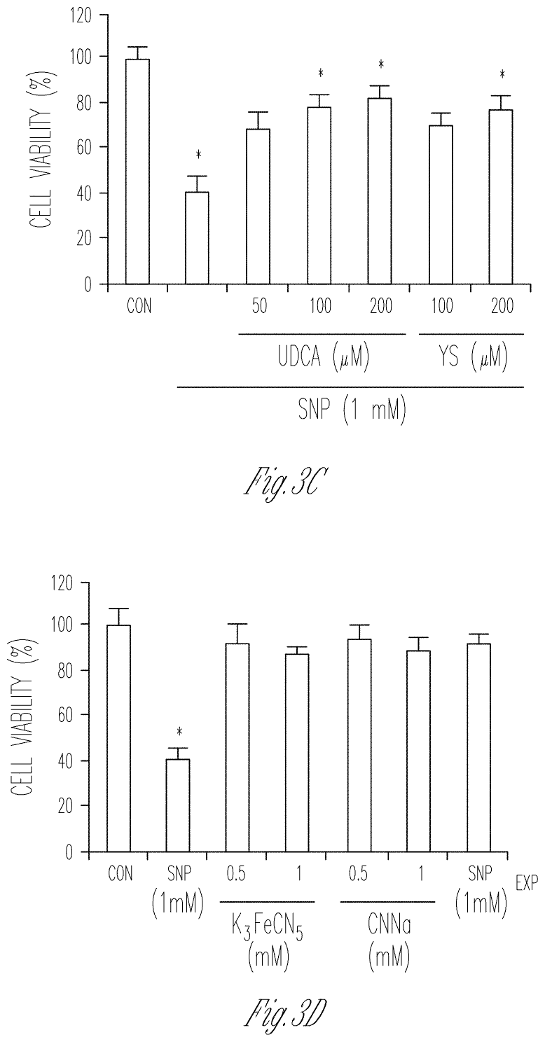

To determine the changes of RNS and ROS production in human dopaminergic cells during the SNP-induced cell death and UDCA- or YS-mediated protection, we measured NO, total ROS, and ONOO⁻ production in SH-SY5Y cells using Griess reagent, fluorescent dye H₂DCFDA, and DHR-123, respectively. NO production after 24 h SNP treatment was increased to 527.74% that of the control group. Both UDCA and YS attenuated the SNP-induced NO production. UDCA pretreatment (50, 100, and 200 μM) dose-dependently reduced the NO production to 91.44%, 82.88%, and 77.26%, respectively, compared with the group treated with SNP alone. Next, we further investigated whether the protective effects of UDCA and YS were due to the decreased production of total ROS and peroxynitrite. Treatment with 1 mM SNP increased total ROS and ONOO⁻ generation up to 324.17% and 174.9%, respectively compared with the control group (FIG. 3A). However, ROS generation was dose-dependently reduced to 79.68%, 72.59%, and 58.09% of SNP-treated group by UDCA pretreatment (50, 100 and 200 μM) and reduced to 76.74% and 66.57% by YS pretreatment (100 and 200 μM), respectively. SNP-induced peroxynitrite generation was inhibited by UDCA (50, 100, and 200 μM) or YS (100 and 200 μM) dose-dependently. Interestingly, pretreatment of cells with high dose of UDCA (200 μM) or YS (200 μM) produced almost complete blocking of SNP-induced peroxynitrie generation.

UDCA and YS Restored the SNP-induced Cellular GSH Content Depletion and Mitochondrial Dysfunction

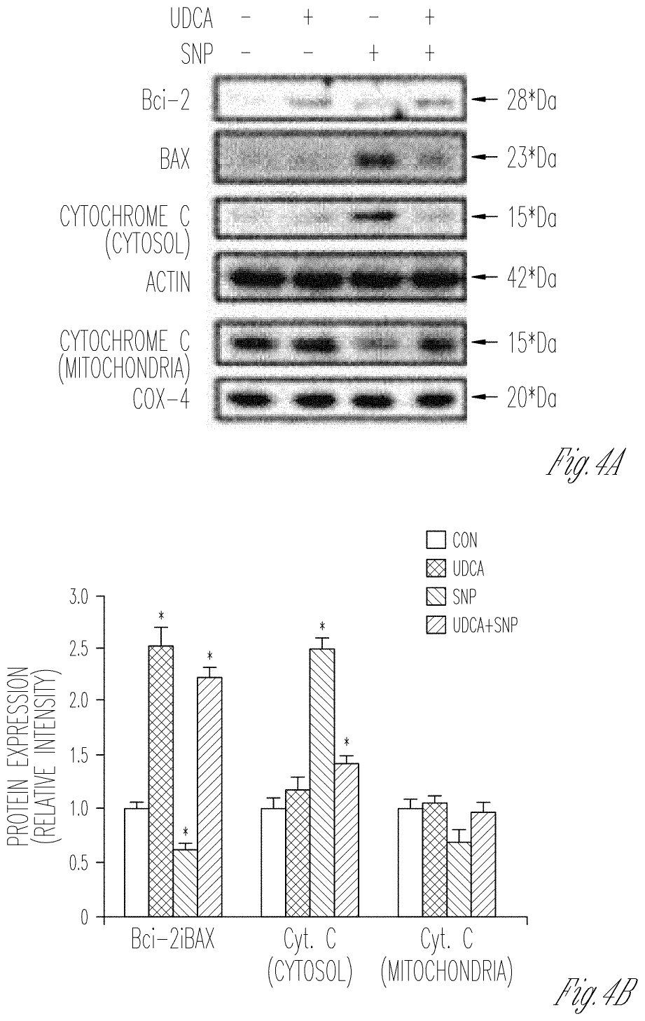

To further evaluate the anti-oxidative effects of UDCA and YS, we determined the levels of intracellular GSH, a major cellular protective antioxidant. As shown in FIG. 4A, cellular GSH level was significantly decreased after treatment with 1 mM SNP for 12 h (49.52+8.4% of control). However, pretreatment with UDCA (50, 100, and 200 μM) or YS (100 and 200 μM) markedly attenuated SNP-induced GSH depletion in SH-SYSY cells.

As shown in FIG. 4B, the control cells and UDCA or YS treated cells did not show any alterations in MMP. Treatment of cells with 1 mM SNP for 12 h significantly decreased MMP to 47% of control group. However, the SNP-induced MMP loss was relieved by UDCA (71%, 88%, and 87% of control group at 50, 100, 200 μM UDCA, respectively) or YS (71% and 74% of control group at 100 and 200 μM YS, respectively).

### UDCA Restored the Bcl-2/Bax Ratio and Prevented the Cytochrome C Release

The mitochondrial dysfunction is accompanied by modulation of Bcl-2 family proteins and release of cytochrome c. To investigate the involvement of Bcl-2 family proteins in SNP-induced cell death and UDCA-mediated protection, we determined the expression of the programmed cell death suppressor protein Bcl-2 and programmed cell death inducer protein Bax by Western blot. SNP treatment showed no alterations in Bcl-2 expression but an increase in Bax expression, which resulted in a decreased ratio of Bcl-2/Bax (0.63±0.05 fold of control). However, UDCA per se and pretreatment with UDCA prior to SNP treatment significantly increased the ratio of Bcl-2/Bax (2.52+0.16 fold and 2.21+0.09 fold of control, respectively) in SH-SYSY cells. In addition, SNP (1 mM) markedly induced cytochrome c release from the mitochondria into the cytosol (2.48+0.11 fold of control). However, the release of cytochrome c was significantly restored (1.41+0.06 fold of control) of pretreatment with UDCA.

UDCA-mediated Neuroprotection is Associated with P13K and Akt/PKB Signal Pathways

To evaluate the signaling pathways in UDCA-mediated neuroprotection against the insult of SNP on SH-SYSY cells, a pharmacological approach was used with specific inhibitors of various signaling molecules. Cells were pretreated with specific Akt/PKB inhibitor triciribine (1 μM), P13K inhibitor LY294002 (2 μM), PKA inhibitor PK1 (1 μM), or PKC inhibitor Go6983 (2 μM) for 1 h, and then treated with UDCA (200 μM) for 1 h and stimulated with SNP (1 mM) for 24 h. However, PM (PKA inhibitor) and Go6983 (PKC inhibitor) did not have significant impact on the UDCA-mediated neuroprotection. All those inhibitors themselves had no effects on cell viability in SH-SYSY cells. To further confirm the role of P13K-Akt/PKB pathways in UDCA-mediated neuroprotection, translocation of the programmed cell death inducer Bax was evaluated after pretreatment with specific inhibitors of P13K and Akt/PKB. Bax translocation to the mitochondria induced by SNP (1 mM) treatment was almost completely blocked by UDCA (200 μM) pretreatment. However, the inhibitory effect of UDCA on SNP-induced Bax translocation was markedly reversed by LY294002 (P13K inhibitor) and triciribine (Akt/PKB inhibitor). These results indicate that UDCA can exert a neuroprotective effect, at least in part, through the P13K-Akt/PKB pathways in SH-SYSY cells.

p53 is a key molecular target of UDCA in regulating apoptosis

p53 plays an important role in regulating expression of genes that mediate cell cycle progression and/or apoptosis. We have previously shown UDCA prevents TGF-131-induced p53 stabilization and apoptosis in primary rat hepatocytes. We therefore hypothesized that p53 may represent an important target in bile acid-induced modulation of apoptosis and cell survival. Functional studies revealed that UDCA reduced both transcriptional and DNA binding activity of p53 tumor suppressor, while promoting its nuclear export in primary rat hepatocytes. These effects led to abrogation of all apoptotic hallmarks induced by p53 overexpression, such as Bax mitochondrial translocation, cytochrome c release and caspase-3 activation. We have also evaluated whether UDCA inhibited p53 via its major repressor, the Mdm-2 protein. Indeed, increased association between p53 and Mdm-2 was detected in hepatocytes preincubated with UDCA. We suggested that by inducing Mdm-2/p53 complex formation, UDCA reduced p53 activity by simultaneously blocking its transactivation domain and enhancing its export to the cytosol. Target knockdown of the mdm-2 gene by posttranscriptional silencing resulted in increased accumulation of p53 in the nucleus, even in the presence of UDCA, thus confirming the specific role of Mdm-2 in the anti-apoptotic function of UDCA.

We have further extended these studies to explore the role of UDCA in downregulating p53 by Mdm-2. The results showed that the bile acid increases cellular proteasomal activity, thereby decreasing p53 half-life. Importantly, after proteasomal inhibition, UDCA pre-treatment resulted in accumulation of Mdm-2-dependent ubiquitinated p53. Finally, the protective effect of UDCA against p53-induced apoptosis was abolished after inhibition of proteasome activity. In conclusion, these findings suggest that UDCA protects cells from p53-induced apoptosis by promoting its degradation via the Mdm-2-ubiquitin-proteasome pathway.

The fact that proteasomal degradation has been described as the main mechanism by which Mdm-2 inhibits p53 prompted us to investigate the role of UDCA in this pathway. Our data indicated that UDCA stimulated Mdm-2-dependent ubiquitination of p53; further increased proteasome activity triggered by wild-type p53. After proteasomal inhibition, UDCA pre-treatment resulted in accumulation of Mdm-2-dependent ubiquitinated p53. Of note, the protective function of UDCA was abolished by inhibiting proteasome activity.

These data suggest that UDCA protects hepatocytes from p53-induced apoptosis by enhancing complex formation between p53 and its inhibitor Mdm-2. Furthermore, by acting as a chaperone-like molecule, UDCA modulate specific and diverse regulatory events such as transcription, subcellular localization, and degradation of precise apoptosis-related molecular targets.

Genomic Profiling of Rat Hepatocytes after Incubation with UDCA by Microarray Analysis

We have investigated the effects of UDCA on gene expression in primary rat hepatocytes by microarray analysis of the rat genome. We determined the global profile of genes regulated by UDCA by using Affymetrix GeneChip® Rat Expression Array 230A, consisting of approximately 16,000 transcripts and variants. cRNA prepared from vehicle-treated cells was used for comparative analysis. The relative levels of gene expression after 24 h treatment of hepatocytes with 100 μM UDCA were compared by plotting the average difference between cells, and determining the fold change in gene expression. Approximately 441 genes (2.76%) exhibited alterations in expression following UDCA treatment, with a greater than 1.5-fold change in genes expression.

Among these, approximately 25% fulfilled the filtering criteria for detection in at least one of the arrays. Of these 96 genes, 28 were up-regulated and 68 were down-regulated. These genes fall into several broad categories, although some of the most prominent are involved in cell cycle/proliferation and apoptosis. For example, the array analysis indicated that Apaf-1 is robustly down-regulated in rat hepatocytes in response to UDCA. We also assessed the specificity and sensitivity of the microarray analysis. Hierarchical clustering was performed using specific gene subsets. As expected, all three controls clustered with remarkable identity and separated from the three UDCA treated samples on the dendrogram.

Our data indicate that UDCA and TUDCA have markedly anti-apoptotic properties. Characterization of the molecular basis for their anti-apoptotic effects will provide significant new information about the events involved in cell death and the potential check points that may promote cell survival. The toxicity of MPTP and 3-NP are closely related. MPTP toxicity is mediated by inhibition of complex I of the electron transport chain, and is preferentially taken up by dopaminergic cells. 3-NP acts by irreversibly inhibiting complex II of the electron transport chain. By impairing mitochondrial function, MPTP and 3-NP both cause depressed oxidative phosphorylation leading to decreased ATP production and mitochondrial stress. We have previously generated extensive data using 3-NP as the primary toxin. However, the similarities between MPTP and 3-NP suggests that TUDCA will affect MPTP toxicity in a manner similar to that of 3-NP.

Design and Synthesis of phosphorylated dopaminergic prodrugs

Included here are alkyl derivatives of L-dopa, monoamine oxidase inhibitors (MAO), catechol-O-methyl transferase (COMT) and the monoamine re-uptake inhibitors. Converting these molecules and their analogs to pro-drugs by conjugating them with phosphorylated bile acids would greatly enhance the transport through the blood brain barrier which currently is a huge challenge.

Glutamate plays a central role in the disruption of normal basal ganglia function, and it has been hypothesized that agents acting to restore normal glutamatergic function may provide therapeutic interventions that bypass the severe motor complications associated with current DA replacement strategies. Analysis of glutamate receptor ligands in the basal ganglia suggests that both ionotropic and metabotropic glutamate receptors could have anti-parkinsonian actions. Delivery of NMDA receptor antagonists that selectively target the NR2B subunit and antagonists of the metabotropic glutamate receptor mGluR5 also may hold promise. For example, amantadine releases DA from nerve endings of brain cells and stimulates norepinephrine response. Importantly, amantadine also relieves levodopa-induced dyskinesia. Conjugates of phosphorylated bile acid prodrugs with amantadine, kinurenic acid, (metabolic product of L-tryptamine), nipecotic acid, isonipacotic acid, will be used for their anti-parkinsonian activity.

Glutathione (GSH) is the most important antioxidant in biological systems. Several strategies have been used to increase GSH as a means to obtain protection against oxidants such as free radicals and reactive electrophiles such as quinones. Glutathione is present at up to 150 mg/day in the human diet and can be absorbed intact in the intestine. Although cysteine that is released from protein degradation can be reutilized for the synthesis of GSH, cysteine is also used for production of taurine and needed for variety of biological functions including detoxification. Oxidative stress evoked by xenobiotics generally result in the depletion of cellular GSH. A current experimental therapy for Parkinson's disease involves intravenous infusion of GSH. The GSH conjugate of the metabolite of the anti-alcohol agent disulfiram (111) and metabolites of amphetamine and metamphetamine readily cross the BBB via a GSH transporter (112). The relevance to our drug design strategy is S-conjugated GSH with UDCA which is expected to be actively transported via GSH or bile acid transporters in the brain when administered intranasally.

In addition to lipoic acid's role as cofactor in the citrate synthase, it is a powerful antioxidant that is effective at scavenging both water and lipid soluble free radicals. It picks up some of the free radicals that vitamin C and E miss. Lipoic acid is emerging as one of the most promising agents for neuroprotection in neurodegenerative diseases. It acts as a metal chelator for ferrous iron, copper, cadmium and also participates in the regulation of endogenous antioxidants. UDCA (and its analogs and derivatives) conjugate of lipoic acid will be used for neuroprotection activity.

Acetyl-L-carnitine has been demonstrated to increase cellular ATP production. It was shown to prevent MPTP-induced neuronal injury in rats. Further, acetyl-L-carnitine reduces production of mitochondrial free radicals, helps maintain transmembrane mitochondrial potential, and enhances NAD/NADH electron transfer. These conjugates of (and its analogs and derivatives) will be used for protection against neuronal injury.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A compound having the formula (I): 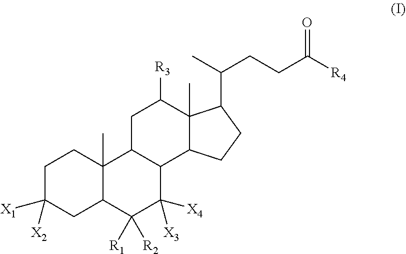
wherein
R1 is —OH, or —(PO4), or -L-Dopa,
R2 is —OH, or —(PO4), or -L-Dopa,
R3 is —OH, —H or —(PO4),
R4 is —OH, —(PO4), or -L-Dopa,
X1 is —H, —OH, —(PO4), or -L-Dopa,
X2 is —H, —OH, —(PO4), or -L-Dopa
X3 is —H, —OH, —(PO4), or -L-Dopa,
X4 is —H, —OH, or -L-Dopa,
wherein at least one of R1, R2, R4, X1, X2, X3, and X4 is -L-Dopa:
or a compound having the formula (II) 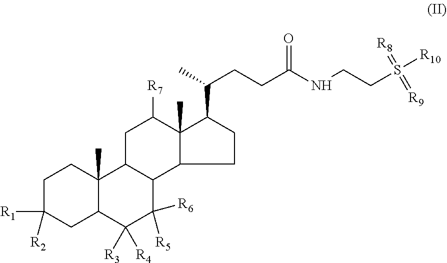
wherein
R1 is —OH, —(PO4), or -L-Dopa,
R2 is —OH, —(PO4), or -L-Dopa,
R3 is —OH, —H or —(PO4),
R4 is —OH, —(PO4), or -L-Dopa,
R5 is —H, —OH, —(PO4), or -L-Dopa,
R6 is —H, —OH, —(PO4), gr -L-Dopa,
R7 is —H, —OH, —(PO4), or -L-Dopa,
R8 is —H, —OH, —(PO4), ═O, or -L-Dopa,
R9 is —H, —OH, —(PO4), ═O, or L-Dopa,
R10 is —H, —OH, —(PO4), or L-Dopa,
wherein at least one of R1, R2, R4, R5, R6, R7, R8, and R9 is -L-Dopa:
or a compound having the formula (ITT) 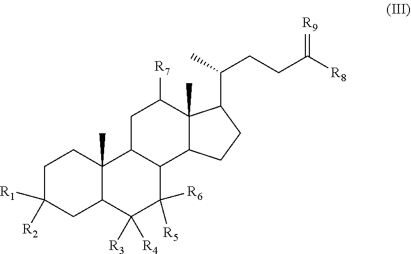
wherein
R1 is —OH, —(PO4), or -L-Dopa,
R2 is —OH, —(PO4), or -L-Dopa,
R3 is —OH, —H or—(PO4),
R4 is —OH, —(PO4), or -L-Dopa,
R5 is —H, —OH, —(PO4), or -L-Dopa,
R6 is —H, —OH, —(PO4), or -L-Dopa,
R7 is —H, —OH, —(PO4), or -L-Dopa,
R8 is —H, —OH, —(PO4), or -L-Dopa,
R9 is —H, —OH, —(PO4), or -L-Dopa,
wherein at least one of R1, R2, R4, R5, R6, R7, R8, and R9 is -L-Dopa;
or a compound having the formula (IV) 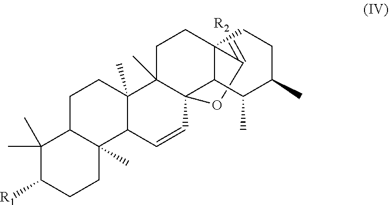
wherein
R1 is —OH, —(PO4), -or -L-Dopa,
R2 is —OH, ═O, —(PO4), or -L-Dopa,
wherein at least one of R1 and R2 is -L-Dopa;

2. A method of retarding Parkinson's disease in a subject, said method comprising administering to said subject a therapeutically effective amount of the compound according to claim 1.

3. A method of retarding a neurological disease in a subject, wherein the disease is selected from the group consisting of Alzheimer's, Huntington's and Amyotrophic lateral sclerosis (ALS), said method comprising administering to said subject a therapeutically effective amount of the compound according to claim 1.

4. (canceled)
